(19) United States
(12) Patent Application Publication
(10) Pub. No.: US 2019/0008867 A1
(43) Pub. Date: Jan. 10, 2019

(54) 4-(4-CYANO-2-THIOARYL)DIHYDROPYRIMIDINONES FOR TREATING CHRONIC WOUNDS

(71) Applicant: pH PHARMA CO., LTD., Seoul (KR)

(72) Inventors: Ekkehard May, Berlin (DE); Stefan Joachim Jodl, Kleinmachnow (DE); Jorn Kratzschmar, Berlin (DE); Volkhart Min-Jian Li, Velbert (DE)

(73) Assignee: pH PHARMA CO., LTD., Seoul (KR)

(21) Appl. No.: 15/775,716

(22) PCT Filed: Nov. 9, 2016
(86) PCT No.: PCT/EP2016/077059

§ 371 Date: May 11, 2018

(30) Foreign Application Priority Data
Nov. 13, 2015 (EP) 15194620.9

(51) Int. Cl. A61K 31/513; A61K 45/06; A61P 17/02

(52) U.S. Cl. CPC A61K 31/513; A61P 17/02; A61K 45/06

(57) ABSTRACT

The invention relates to 4-(4-Cyano-2-thioaryl)dihydropyrimidinones of the formula (I) known from WO 2009/080199(A1) for use in a method for the treatment and/or recurrence rate reduction of a chronic wound selected from different types of ulcers and a chronic wound associated with Behçet's disease wherein the compound of the formula (I) is administered orally and wherein the treatment and/or reduction of recurrence rate of the chronic wound causes one or more of the effects selected from an increased wound closure rate, a reduced wound size, a shorter time to wound closure, an increase of the reepithelialisation of the chronic wound, an increase of the deposition of extracellular matrix such as collagen in the chronic wound, and a reduction of pain related to the chronic wound. The invention further relates to compounds of the formula (I) for use in a method for the treatment of neutrophilic dermatoses or for use in a method for the treatment of autoimmune blistering dermatoses.

The invention relates to 4-(4-Cyano-2-thioaryl)dihydropyrimidinones of the formula (I) known from WO 2009/080199(A1) for use in a method for the treatment and/or recurrence rate reduction of a chronic wound selected from different types of ulcers and a chronic wound associated with Behçet's disease, wherein the treatment and/or reduction of recurrence rate of the chronic wound causes one or more of the effects selected from an increased wound closure rate, a reduced wound size, a shorter time to wound closure, an increase of the reepithelialisation of the chronic wound, an increase of the deposition of extracellular matrix (ECM) such as collagen in the chronic wound, and a reduction of pain related to the chronic wound. The invention further relates to compounds of the formula (I) for use in a method for the treatment of neutrophilic dermatoses or for use in a method for the treatment of autoimmune blistering dermatoses.

Human leukocyte elastase (HLE, EC 3.4.21.37), also called human neutrophil elastase (HNE, hNE), belongs to the family of the serine proteases. The proteolytic enzyme is found in the azurophilic granules of polymorphonuclear leukocytes (PMN leukocytes). Intracellular elastase performs an important function in defense against pathogens by breaking down the foreign particles taken by phagocytosis. Activated neutrophilic cells release the HNE from the granules into the extracellular space (extracellular HNE), with some of the released HNE remaining on the outside of the neutrophilic cell membrane (membrane-associated HNE). The highly active enzyme is able to break down a large number of connective tissue proteins, for example the proteins elastin, collagen and fibronectin. Elastin occurs in high concentrations in all tissue types showing high elasticity, for example in the lung and the arteries. HNE is involved in the tissue breakdown and transformation (tissue remodeling) associated with a large number of pathological processes (for example tissue injuries). HNE is also an important modulator of inflammatory processes. HNE induces for example increased interleukin-8 (IL-8) gene expression.

Accordingly, it is presumed that HNE plays an important role in many disorders, injuries and pathological changes whose formation and/or progression are/is associated with inflammatory events and/or proliferative and hypertrophic tissue, vessel transformation and the breakdown of growthfactors and ECM.

It is generally assumed that elastase-mediated pathological processes are based on a displaced equilibrium between free elastase and endogenous elastase inhibitor protein (mainly alpha-1 antitrypsin, AAT) [Nezarophils and protease/antiprotease imbalance, Stockley, Am. J. Respir. Crit. Care Med. 160, 49-52 (1999)]. AAT is present in large excess in the plasma and thus very rapidly neutralizes free HNE. The concentration of free elastase is elevated in various pathological processes, so that there is a local shift in the balance between protease and protease inhibitor in favor of the protease. In addition, membrane-associated elastase of the activated PMN cells is very substantially protected from inhibition by AAT. The same applies to free elastase, which is located in a microcompartment which is difficult to access between the neutrophilic cell and the adjoining tissue cell (for example endothelial cell). In addition, strong oxidizing conditions prevail in the vicinity of activated leukocytes (oxidative burst), and thus AAT is oxidized and loses several orders of magnitude in the inhibitory effect.

Elastase-inhibiting active compounds (exogenously administered inhibitors of HNE) ought accordingly to have a low molecular weight in order to be able also to reach and inhibit the membrane-associated HNE and the HNE present in the protected microcompartment (see above). Also necessary for this purpose is good in vivo stability of the substances (low in vivo clearance). In addition, these compounds ought to be stable under oxidative conditions in order not to lose inhibitory power in the pathological process.

In Japan and South Korea, an elastase inhibitor (sivelestat, Elaspol®) is approved for the treatment of acute lung injury associated with SIRS. The reversible, but reactive compound has only a relatively weak effect on HNE (Kᵢ 200 nM) and also acts on the pancreas elastase (IC₅₀ 5.6 μM). The active compound is administered intravenously, oral administration is not possible.

Elafin and structural analogs are also investigated as therapeutically useful elastase inhibitors. Elafin is an endogenous small protein which inhibits both elastase and proteinase 3. However, owing to the proteinergic character, oral administration of elafin is not possible.

As disclosed in WO 2009/080199 (A1), it has been found that 1,4-diaryldihydropyrimidin-2-one derivatives are particularly suitable for the treatment and/or prevention of disorders. These compounds described below are low-molecular-weight, non-reactive and selective inhibitors of human neutrophil elastase (HNE) which, surprisingly, show considerably better inhibition of this protease than the compounds known from the prior art. In addition, the compounds disclosed in WO 2009/080199 (A1) have unexpectedly low in vitro clearance in hepatocytes and thus improved metabolic stability.

WO 2010/115548 (A1) relates to sulfonamide- or sulfoximine-substituted 1,4-diaryldihydropyrimidin-2-one derivatives as inhibitors of human neutrophil elastase (HNE) and their use for the treatment and/or prevention of diseases, in particular for the treatment and/or prevention of disorders of the lung and the cardiovascular system. The compounds disclosed in WO 2010/115548 (A1) differ from the compounds for use in the present invention at least in the substituent Z, since sulfonamide- or sulfoximine-substituents are not comprised by the formula (I) of WO 2009/080199 (A1).

The disorders, injuries and pathological changes related to HNE further include chronic wounds, wound related pain and neuropathic pain (Repurposing a leukocyte elastase inhibitor for neuropathic pain, Andy D Weyer et al., Nature Medicine 21, 429-430 (2015)).

All wound types have the potential to become chronic and, as such, chronic wounds are traditionally divided etiologically. Identifying and treating the underlying aetiology of a chronic wound such as venous insufficiency, postthrombotic syndrome (PTS), disturbance of the arterial perfusion (critical limb ischemia), diabetes, postsurgical complications, or unrelieved pressure as well as systemic factors such as nutritional status, immuno suppression, AAT-insufficiency and/or infections that may contribute to poor wound healing are key to successful wound treatment. The most commonly encountered chronic wound is the lower extremity ulcer; these are generally vascular or diabetic in nature and account for up to 98% of all lower extremity wounds (Werdin, Evidence-based Management Strategies for Treatment of Chronic Wounds, ePlasty, 2009; 9: e19, 2009: 169-179).

In 2011 there were 350 million diabetics world-wide 6.6% of the population), and this number is expected to double until 2028. Diabetic foot ulcers are the most frequent cause of hospitalisations of diabetics. The risk of a diabetic to develop diabetic foot ulcer in his or her lifetime is 15-25%, 15% of all diabetic foot ulcers lead to amputation. World-wide, 40-70% of all non-traumatic amputations are carried out on diabetics. Risk factors for diabetic foot ulcers are traumata, poor metabolic control, sensory, motoric and autonomous polyneuropathy, inappropriate footwear, infections and peripheral arterial disorders. The treatment and/or recurrence rate reduction of diabetic foot ulcers requires interdisciplinary teams and employs a multifactor approach: weight loss, revascularisation (in the case of peripheral arterial occlusive disease, PAOD), improvements in metabolic control, wound debridement, wound stage adapted dressings, dalteparin, Regranex (PDGF) and eventual amputation. The treatment costs per diabetic foot ulcer (without amputation) are 7,000-10,000 USD. 33% of all diabetic foot ulcers do not heal within 2 years, and there is a high recurrence rate (34% within the first year, 61% over 3 years).

Neutrophilic dermatoses are a heterogenous group of diseases that share the overactivation of neutrophilic granulocytes as underlying pathophysiological factor. Chronic wounds associated with neutrophilic dermatoses include chronic wounds caused by pyoderma gangrenosum (PG) or Behçet syndrome.

The success of wound therapy is reflected by and may be assessed via an increased wound closure rate, a reduced wound size, a shorter time to wound closure, an increase of the reepithelialisation of the chronic wound, an increase of the deposition of extracellular matrix such as collagen in the chronic wound, and a reduction of pain related to the chronic wound.

Epithelialization is an essential component of wound healing, used as a defining parameter of a successful wound closure. A wound cannot be considered healed in the absence of reepithelialization. The epithelialization process is impaired in all types of chronic wounds. Epithelialization is an essential component of wound healing used as a defining parameter of its success. In the absence of reepithelialization, a wound cannot be considered healed. Barrier breach provides a portal for wound infection. This process is impaired in all types of chronic wounds. Failure of keratinocytes to maintain the barrier may contribute to wound reoccurrence, which is another significant clinical problem. A better understanding of the epithelialization process may provide insights for new therapeutic approaches to accelerate wound closure. (Epithelialization in Wound Healing: A Comprehensive Review, Pastar et al., ADVANCES IN WOUND CARE, 2014, VOLUME 3, NUMBER 7, 445-464)

Topical wound care remains the Standard of Care (SoC) treatment of chronic wounds for the time being with a big variety of different mechanisms and technologies such as gaze, hydrocolloidal wound covers, foams, gels etc. The respective topical treatment needs to be chosen and applied according to the actual healing stage of the chronic wound. In addition (mechanical) debridement, measure to optimize the vascular status and surgical options with skin transplantations may be considered (see also below).

Except systemic antibiotic treatment that is not aiming to improve wound healing but to control wound borne infection, various approaches to treat chronic wounds via systemic exposure have yielded no or only limited success so far. Oral Aspirin was tried to address pain and inflammation associated with chronic wounds (de Oliveira Carvalho, Cochrane Database Syst Rev. 2 (2016) Art. No. CD009432, 4 pages, 2016) while oral zinc supplementation targeted inflammatory and proliferative stages (Bradbury, Clin Rev Wounds 2006, 2: 54-61). Both approaches remained inconclusive. Significant effort had been invested to explore beneficial effects of Pentoxifylline on wound healing. A metaanalysis (Jull, Lancet 359 (2002) pp. 1550-1554) suggests moderate efficacy, however, the overall study results were non-uniform. Consequently, even though a systemic drug would be highly appreciated by physicians and patients, neither Pentoxifylline nor any other oral drug was ever approved for the treatment of chronic wounds.

There remain thus unmet needs with regard to the therapy of chronic wounds, such as the need for oral therapy.

The invention provides a compound of the formula (I)

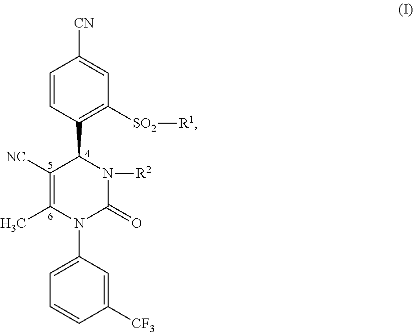

in which
R¹ represents (C₁-C₄)-alkyl and
R² represents hydrogen, (C₁-C₂)-alkyl or a group of the formula —CH₂—C(═O)—NH—R³ or —SO₂—R⁴, in which
   R³ represents hydrogen or (C₁-C₂)-alkyl and
   R⁴ represents (C₁-C₂)-alkyl or (C₃-C₄)-cycloalkyl,

or a salt, a solvate or a solvate of a salt thereof,

for use in a method for the treatment and/or recurrence rate reduction of a chronic wound selected from the group consisting of a pressure ulcer, a diabetic ulcer on the extremities, a venous leg ulcer, an arterial leg ulcer, a mixed leg ulcer, and a chronic wound associated with Behçet's disease, wherein the compound of the formula (I) is administered orally and wherein the treatment and/or reduction of recurrence rate of the chronic wound causes one or more of the effects selected from an increased wound closure rate, a reduced wound size, a shorter time to wound closure, an increase of the reepithelialisation of the chronic wound, an increase of the deposition of extracellular matrix such as collagen in the chronic wound, and a reduction of pain related to the chronic wound.

The invention further provides a compound as defined above for use in a method for the treatment of neutrophilic dermatoses selected from Behçet's disease, PAPA-syndrome, PASH syndrome, SAPHO syndrome, and subcorneal pustular dermatosis or for use in a method for the treatment of autoimmune blistering dermatoses.

The invention further provides a compound as defined above for use in a method for the treatment of autoimmune blistering dermatoses, wherein the autoimmune blistering dermatoses are selected from the group consisting of pemphigus, bullous pemphigoid, epidermolysis bullosa acquisita, mucous membrane pemphigoid, pemphigoid gestationis, linear IgA dermatosis, and dermatitis herpetiformis.

The compounds for use in the methods of the invention and their synthesis are known from WO 2009/080199.

A chronic wound, also termed chronic cutaneous ulcer, within the meaning of the present invention is a wound that has failed to proceed through an orderly and timely series of events to produce a durable structural, functional, and cosmetic closure over a period of three months (Guidance for Industry Chronic Cutaneous Ulcer and Burn Wounds, FDA Wound Healing Clinical Focus Group, Wound Repair and Regeneration 2001, Vol 9, No. 4:258-268).

Wound closure within the meaning of the present invention is defined as closure of the skin defect/ulcer with complete reepithelialization.

An increased wound closure rate within the meaning of the present invention is defined as a wound closure rate within 12-16 weeks of treatment according to the invention in % increase over placebo, for example more than 10%, more than 15%, more than 20%, more than 25%, more than 30%, more than 40%, more than 50%, more than 60%, more than 70%, more than 80%, or more than 90% increased wound closure rate in relation to placebo.

According to a further embodiment of the invention, increased wound closure rates are defined as a wound closure in % within 12-16 weeks of treatment according to the invention in addition to standard of care treatment that is increased over standard of care treatment alone, for example more than 10%, more than 15%, more than 20%, more than 25%, more than 30%, more than 40%, more than 50%, more than 60%, more than 70%, more than 80%, or more than 90% increased wound closure rates in relation to standard of care alone.

Standard of care (SoC) within the meaning of the present invention is defined as physical, biological, topical and/or systemic wound management therapies selected from the group consisting of topical wound dressings, topical antiseptics, wound excision or debridement, weight reduction, appropriate footwear for an offloading effect, PDGF (Regranex), hyperbaric oxygen therapy, compression therapy, wound therapy with negative pressure, maggot debridement therapy, and therapy with systemic antibiotics.

A reduced wound size within the meaning of the present invention is defined as wound size at a certain time after start of treatment according to the invention in % of the wound size at day 0 of treatment. Examples are a wound size at a certain time after start of treatment of less than 90%, less than 80%, less than 70%, less than 60%, less than 50%, less than 40%, less than 30%, or less than 20% of the wound size at day 0 of treatment.

The reduced wound size (or wound area reduction, WAR) may be assessed within a timeframe of e.g. 8-16 weeks. The reduced wound size (or WAR) is considered a relevant parameter, indicating a treatment effect, since WAR is regarded as reliable predictor of later complete wound closure (Cardinal M E, Harding K et al, Wound Rep Reg (2008) 16 19-22).

Within the meaning of the present invention, the term “reduced wound size” is used synonymously with the term “wound area reduction (WAR)”.

A shorter time to wound closure within the meaning of the present invention is defined as the time from the start of the treatment according to the invention until complete closure of the wound in % of placebo, for example less than 90%, less than 80%, less than 70%, less than 60%, less than 50%, less than 40%, less than 30%, less than 20%, less than 10% of the wound closure time observed in placebo.

According to a further embodiment of the invention a shorter time to wound closure within the meaning of the present invention is defined as the time from the start of the treatment according to the invention in addition to standard of care treatment until complete closure of the wound in % of standard of care alone, for example less than 90%, less than 80%, less than 70%, less than 60%, less than 50%, less than 40%, less than 30%, less than 20%, less than 10% of the wound closure time observed with standard of care alone.

An increase of the reepithelialisation of the chronic wound within the meaning of the present invention is defined as the restauration of the upper epidermal/keratinocyte layer restoring the skin barrier for protection against external physical factors or pathological factors auch as bacteria or other pathogens. Epithelialisation is the last step in the cascade of wound healing.

An increase of wound closure (reepithelialisation) of the chronic wound within the meaning of the present invention is defined as for example a wound closure (reepithelialisation) at a certain time from the start of the treatment according to the invention of more than 80%, more than 85%, more than 90%, more than 95%, more than 99%, more than 99.5% or 100% of the initial epidermal gap.

An increase of the deposition of extracellular matrix in the chronic wound within the meaning of the present invention is defined as for example an increase of newly synthesized collagen type III deposition in long lasting skin lesions vs. collagen I (old collagen) at a certain time from the start of the treatment according to the invention in relation to placebo by for example more than 10%, more than 15%, more than 20%, more than 25%, more than 30%, more than 40%, more than 50%, more than 60%, more than 70%, more than 80%, or more than 90% over placebo.

According to a further embodiment of the invention an increase of the deposition of extracellular matrix in the chronic wound is defined as for example an increase of newly synthesized collagen type III deposition in long lasting skin lesions vs. collagen I (old collagen) at a certain time from the start of the treatment according to the invention in addition to standard of care treatment in relation to standard of care treatment alone by for example more than 10%, more than 15%, more than 20%, more than 25%, more than 30%, more than 40%, more than 50%, more than 60%, more than 70%, more than 80%, or more than 90% over standard of care treatment alone.

A reduction of the recurrence rate of a chronic wound within the meaning of the present invention is defined as a recurrence rate within 12 weeks after wound closure following treatment according to the invention that is below that of placebo, for example a recurrence rate that is more than 10%, more than 15%, more than 20%, more than 25%, more than 30%, more than 40%, more than 50%, more than 60%, more than 70%, more than 80%, or more than 90% lower than the recurrence rate of placebo.

According to a further embodiment of the invention a reduction of the recurrence rate of a chronic wound is defined as a recurrence rate within 12 weeks after wound closure following treatment according to the invention in addition to standard of care treatment that is below that of standard of care treatment alone, for example a recurrence rate that is more than 10%, more than 15%, more than 20%, more than 25%, more than 30%, more than 40%, more than 50%, more than 60%, more than 70%, more than 80%, or more than 90% lower than the recurrence rate of standard of care treatment alone.

Reduction of pain related to the chronic wound within the meaning of the current invention is defined as reduction of painful sensations that may be assessed by a Visual Analogue Scale (VAS) and/or by patient or physician reported pain diaries or indirectly by the reduction of the use or of the amount or of the strength of analgesic drugs.

Compounds for use in the method of the invention are the compounds of the formula (I) and the salts, solvates and solvates of the salts thereof, and also the compounds encompassed by formula (I) and specified hereinafter as specific example(s), and the salts, solvates and solvates of the salts thereof, to the extent that the compounds encompassed by formula (I) and specified hereinafter are not already salts, solvates and solvates of the salts. The structure and the synthesis of the compounds of formula (I) are known from WO 2009/080199.

The compounds for use in the method of the invention may, depending on their structure, exist in different stereoisomeric forms, i.e. in the form of configurational isomers or else optionally as conformational isomers (enantiomers and/or diastereomers, including those in the case of atropisomers). The present invention therefore encompasses the enantiomers and diastereomers, and the respective mixtures thereof. The stereoisomerically uniform constituents can be isolated from such mixtures of enantiomers and/or diastereomers in a known manner; chromatography processes are preferably used for this, especially HPLC chromatography on an achiral or chiral phase.

Where the compounds for use in the method of the invention can occur in tautomeric foul s, the present invention encompasses all the tautomeric forms.

The present invention also encompasses the use in the method of the invention of all suitable isotopic variants of the compounds of formula (I). An isotopic variant of a compound for use in the method of the invention is understood here as meaning a compound in which at least one atom within the compound according to the invention has been exchanged for another atom of the same atomic number, but with a different atomic mass than the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸P, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active compound distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example to an extension of the half-life in the body or to a reduction in the active dose required; such modifications of the compounds according to the invention may therefore in some cases also constitute a preferred embodiment of the present invention. Isotopic variants of the compounds according to the invention can be prepared by the processes known to those skilled in the art, for example by the methods described below and the procedures described in the working examples, by using corresponding isotopic modifications of the respective reagents and/or starting compounds.

Preferred salts of the compounds for use in the method according to the invention are physiologically acceptable salts of the compounds according to the invention. The invention also encompasses salts which themselves are unsuitable for pharmaceutical applications but which can be used, for example, for the isolation or purification of the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

Solvates in the context of the invention are described as those forms of the compounds according to the invention which form a complex in the solid or liquid state by coordination with solvent molecules. Hydrates are a specific form of the solvates in which the coordination is with water.

In addition, the present invention also encompasses prodrugs of the compounds according to the invention. The term “prodrugs” includes compounds which may themselves be biologically active or inactive but are converted to compounds according to the invention while resident in the body (for example metabolically or hydrolytically).

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “reduction of recurrence rate”, “reduction of reoccurrence rate”, and “reduction of relapse rate” are used synonymously in the context of the present invention and refer to reduction of the risk that a wound that showed complete healing relapses or recurs.

The terms “prophylaxis of recurrence (or reoccurrence, or relapse)” and “preclusion of recurrence (or reoccurrence, or relapse)” of a chronic wound are also used synonymously in the context of the present invention and refer to preventing the relapse or reoccurrence of a wound that showed complete healing.

The treatment or prevention of a disease, a condition, a disorder, an injury or a health problem may be partial or complete.

In the context of the present invention, unless specified otherwise, the substituents of the compounds of formula (I) are defined as follows:

According to an embodiment of the present invention, the compounds of the formula (I) for use in a method for the treatment and/or recurrence rate reduction of a chronic wound selected from the group consisting of a pressure ulcer, a diabetic ulcer on the extremities, a venous leg ulcer, an arterial leg ulcer, a mixed leg ulcer, and a chronic wound associated with Behçet's disease, wherein the compound of the formula (I) is administered orally and wherein the treatment and/or recurrence rate reduction of the chronic wound causes one or more of the effects selected from increased wound closure rates, reduced wound size, shorter time to wound closure, an increase of the reepithelialisation of the chronic wound, an increase of the deposition of extracellular matrix such as collagen in the chronic wound, and a reduction of pain related to the chronic wound are defined as follows:
R¹ represents (C₁-C₂)-alkyl and
R² represents hydrogen, methyl or a group of the formula —CH₂—C(═O)—NH—R³ or —SO₂—R⁴, in which
   R³ represents hydrogen or methyl and
   R⁴ represents methyl or cyclopropyl,

or a salt, a solvate or a solvate of a salt thereof.

According to an embodiment of the present invention, the compounds of the formula (I) for use in a method for the treatment of neutrophilic dermatoses selected from Behçet's disease, PAPA-syndrome, PASH syndrome, SAPHO syndrome, and subcorneal pustular dermatosis or for use in a method for the treatment of autoimmune blistering dermatoses, are defined as follows:
R¹ represents (C₁-C₂)-alkyl and
R² represents hydrogen, methyl or a group of the formula —CH₂—C(═O)—NH—R³ or —SO₂—R⁴, in which
   R³ represents hydrogen or methyl and
   R⁴ represents methyl or cyclopropyl,

or a salt, a solvate or a solvate of a salt thereof.

According to an embodiment of the present invention, the compounds of the formula (I) for use in a method for the treatment and/or recurrence rate reduction of a chronic wound selected from the group consisting of a pressure ulcer, a diabetic ulcer on the extremities, a venous leg ulcer, an arterial leg ulcer, a mixed leg ulcer, and a chronic wound associated with Behçet's disease, wherein the compound of the formula (I) is administered orally and wherein the treatment and/or recurrence rate reduction of the chronic wound causes one or more of the effects selected from increased wound closure rates, reduced wound size, shorter time to wound closure, an increase of the reepithelialisation of the chronic wound, an increase of the deposition of extracellular matrix such as collagen in the chronic wound, and a reduction of pain related to the chronic wound are selected from the group consisting of
(4S)-4-[4-Cyano-2-(methylsulfonyl)phenyl]-6-methyl-2-oxo-1-[3-(trifluoromethyl)phenyl]-1,2,3,4-tetrahydropyrimidine-5-carbonitrile (disclosed in WO 2009/080199 A1 as example 6),
2-[(6S)-5-Cyano-6-[4-cyano-2-(methylsulfonyl)phenyl]-4-methyl-2-oxo-3-[3-(trifluoromethyl)phenyl]-3,6-dihydropyrimidin-1(2H)-yl]acetamide (disclosed in WO 2009/080199 A1 as example 22),
(4S)-4-[4-Cyano-2-(methylsulfonyl)phenyl]-6-methyl-3-(methylsulfonyl)-2-oxo-1-[3-(trifluoromethyl)-phenyl]-1,2,3,4-tetrahydropyrimidine-5-carbonitrile (disclosed in WO 2009/080199 A1 as example 27),
(4S)-4-[4-Cyano-2-(methylsulfonyl)phenyl]-6-methyl-3-(cyclopropylsulfonyl)-2-oxo-1-[3-(tri-fluoromethyl)phenyl]-1,2,3,4-tetrahydropyrimidine-5-carbonitrile (disclosed in WO 2009/080199 A1 as example 32),
(4S)-4-[4-Cyano-2-(methylsulfonyl)phenyl]-3,6-dimethyl-2-oxo-1-[3-(trifluoromethyl)phenyl]-1,2,3,4-tetrahydropyrimidine-5-carbonitrile (disclosed in WO 2009/080199 A1 as example 33),
(4S)-4-[4-Cyano-2-(ethylsulfonyl)phenyl]-3,6-dimethyl-2-oxo-1-[3-(trifluoromethyl)phenyl]-1,2,3,4-tetrahydropyrimidine-5-carbonitrile (disclosed in WO 2009/080199 A1 as example 128), and

or a salt, a solvate or a solvate of a salt thereof.

According to an embodiment of the present invention, the compounds of the formula (I) for use in a method for the treatment of neutrophilic dermatoses selected from Behçet's disease, PAPA-syndrome, PASH syndrome, SAPHO syndrome, and subcorneal pustular dermatosis or for use in a method for the treatment of autoimmune blistering dermatoses, are are selected from the group consisting of:
(4S)-4-[4-Cyano-2-(methylsulfonyl)phenyl]-6-methyl-2-oxo-1-[3-(trifluoromethyl)phenyl]-1,2,3,4-tetrahydropyrimidine-5-carbonitrile (disclosed in WO 2009/080199 A1 as example 6),
2-[(6S)-5-Cyano-6-[4-cyano-2-(methylsulfonyl)phenyl]-4-methyl-2-oxo-3-[3-(trifluoromethyl)phenyl]-3,6-dihydropyrimidin-1(2H)-yl]acetamide (disclosed in WO 2009/080199 A1 as example 22),
(4S)-4-[4-Cyano-2-(methylsulfonyl)phenyl]-6-methyl-3-(methylsulfonyl)-2-oxo-1-[3-(trifluoromethyl)-phenyl]-1,2,3,4-tetrahydropyrimidine-5-carbonitrile (disclosed in WO 2009/080199 A1 as example 27),
(4S)-4-[4-Cyano-2-(methylsulfonyl)phenyl]-6-methyl-3-(cyclopropylsulfonyl)-2-oxo-1-[3-(tri-fluoromethyl)phenyl]-1,2,3,4-tetrahydropyrimidine-5-carbonitrile (disclosed in WO 2009/080199 A1 as example 32),
(4S)-4-[4-Cyano-2-(methylsulfonyl)phenyl]-3,6-dimethyl-2-oxo-1-[3-(trifluoromethyl)phenyl]-1,2,3,4-tetrahydropyrimidine-5-carbonitrile (disclosed in WO 2009/080199 A1 as example 33),
(4S)-4-[4-Cyano-2-(ethylsulfonyl)phenyl]-3,6-dimethyl-2-oxo-1-[3-(trifluoromethyl)phenyl]-1,2,3,4-tetrahydropyrimidine-5-carbonitrile (disclosed in WO 2009/080199 A1 as example 128), and

or a salt, a solvate or a solvate of a salt thereof.

According to an embodiment of the present invention, the compounds of the formula (I) for use in a method for the treatment and/or recurrence rate reduction of a chronic wound selected from the group consisting of a pressure ulcer, a diabetic ulcer on the extremities, a venous leg ulcer, an arterial leg ulcer, a mixed leg ulcer, and a chronic wound associated with Behçet's disease, wherein the compound of the formula (I) is administered orally and wherein the treatment and/or recurrence rate reduction of the chronic wound causes one or more of the effects selected from increased wound closure rates, reduced wound size, shorter time to wound closure, an increase of the reepithelialisation of the chronic wound, an increase of the deposition of extracellular matrix such as collagen in the chronic wound, and a reduction of pain related to the chronic wound are selected from the group consisting of
(4S)-4-[4-Cyano-2-(methylsulfonyl)phenyl]-6-methyl-3-(methylsulfonyl)-2-oxo-1-[3-(trifluoromethyl)-phenyl]-1,2,3,4-tetrahydropyrimidine-5-carbonitrile and
(4S)-4-[4-Cyano-2-(methylsulfonyl)phenyl]-3,6-dimethyl-2-oxo-1-[3-(trifluoromethyl)phenyl]-1,2,3,4-tetrahydropyrimidine-5-carbonitrile,

or a salt, a solvate or a solvate of a salt thereof.

According to an embodiment of the present invention, the compounds of the formula (I) for use in a method for the treatment of neutrophilic dermatoses selected from Behçet's disease, PAPA-syndrome, PASH syndrome, SAPHO syndrome, and subcorneal pustular dermatosis or for use in a method for the treatment of autoimmune blistering dermatoses, are are selected from the group consisting of:
(4S)-4-[4-Cyano-2-(methylsulfonyl)phenyl]-6-methyl-3-(methylsulfonyl)-2-oxo-1-[3-(trifluoromethyl)-phenyl]-1,2,3,4-tetrahydropyrimidine-5-carbonitrile and
(4S)-4-[4-Cyano-2-(methylsulfonyl)phenyl]-3,6-dimethyl-2-oxo-1-[3-(trifluoromethyl)phenyl]-1,2,3,4-tetrahydropyrimidine-5-carbonitrile,

or a salt, a solvate or a solvate of a salt thereof.

The compounds for use according to the present invention and the synthesis thereof are known from WO 2009/080199(A1). The IC₅₀ data for the inhibition of human neutrophil elastase (HNE) and the description of the corresponding assay are known from chapter B-1 and Table A of WO 2009/080199(A1):

| | | Inhibition of human |
|---|---|---|
| Example | | neutrophil elastase (HNE) |
| No. | IUPAC Name | IC₅₀ [nM] |
| 1 | (4S)-4-[4-Cyano-2-(methylsulfonyl)phenyl]-6-methyl-2- | 0.5 |
| | oxo-1-[3-(trifluoromethyl)phenyl]-1,2,3,4- | |
| | tetrahydropyrimidine-5-carbonitrile | |
| 2 | 2-[(6S)-5-Cyano-6-[4-cyano-2-(methylsulfonyl)phenyl]- | 0.45 |
| | 4-methyl-2-oxo-3-[3-(trifluoromethyl)phenyl]-3,6- | |
| | dihydropyrimidin-1(2H)-yl]acetamide | |
| 3 | (4S)-4-[4-Cyano-2-(methylsulfonyl)phenyl]-6-methyl-3- | <0.3 |
| | (methylsulfonyl)-2-oxo-1-[3-(trifluoromethyl)phenyl]- | |
| | 1,2,3,4-tetrahydropyrimidine-5-carbonitrile | |
| 4 | (4S)-4-[4-Cyano-2-(methylsulfonyl)phenyl]-6-methyl-3- | <0.3 |
| | (cyclopropylsulfonyl)-2-oxo-1-[3-(trifluoromethyl) | |
| | phenyl]-1,2,3,4-tetrahydropyrimidine-5-carbonitrile | |
| 5 | (4S)-4-[4-Cyano-2-(methylsulfonyl)phenyl]-3,6- | <0.3 |
| | dimethyl-2-oxo-1-[3-(trifluoromethyl)phenyl]-1,2,3,4- | |
| | tetrahydropyrimidine-5-carbonitrile | |
| 6 | (4S)-4-[4-Cyano-2-(ethylsulfonyl)phenyl]-3,6-dimethyl- | <0.3 |
| | 2-oxo-1-[3-(trifluoromethyl)phenyl]-1,2,3,4- | |
| | tetrahydropyrimidine-5-carbonitrile | |

According to an embodiment of the present invention, the compound of the formula (I) for use in a method for the treatment and/or recurrence rate reduction of a chronic wound selected from the group consisting of a pressure ulcer, a diabetic ulcer on the extremities, a venous leg ulcer, an arterial leg ulcer, a mixed leg ulcer, and a chronic wound associated with Behçet's disease, wherein the compound of the formula (I) is administered orally and wherein the treatment and/or recurrence rate reduction of the chronic wound causes one or more of the effects selected from increased wound closure rates, reduced wound size, shorter time to wound closure, an increase of the reepithelialisation of the chronic wound, an increase of the deposition of extracellular matrix such as collagen in the chronic wound, and a reduction of pain related to the chronic wound is (4S)-4-[4-Cyano-2-(methylsulfonyl)phenyl]-3,6-dimethyl-2-oxo-1-[3-(trifluoro methyl)phenyl]-1,2,3,4-tetrahydropyrimidine-5-carbonitrile or a salt, a solvate or a solvate of a salt thereof.

According to an embodiment of the present invention, the compound of the formula (I) for use in a method for the treatment of neutrophilic dermatoses selected from Behçet's disease, PAPA-syndrome, PASH syndrome, SAPHO syndrome, and subcorneal pustular dermatosis or for use in a method for the treatment of autoimmune blistering dermatoses is (4S)-4-[4-Cyano-2-(methylsulfonyl)phenyl]-3,6-dimethyl-2-oxo-1-[3-(trifluoromethyl)phenyl]-1,2,3,4-tetrahydropyrimidine-5-carbonitrile or a salt, a solvate or a solvate of a salt thereof.

Surprisingly, the compounds according to the present invention were found to be effective for the treatment and/or recurrence rate reduction of a chronic wound when given orally. For example, as shown in table 5, linear increase of exposure was found in blood plasma as well as in skin (both in native as well as wounded skin). This correlated well with an inhibition of NE activity in wound tissue which in turn correlated with an improved visual wound size reduction. Table 7 shows that a compound according to the invention potently and effectively inhibited myeloperoxidase activity in wound tissue already at a low dose of 0.1 mg/kg. Myeloperoxidase activity was measured to assess neutrophil activity in wound tissue samples.

One embodiment of the present invention is also a compound of the formula (I) as defined above for use in the method for the treatment and/or recurrence rate reduction of a chronic wound according to the invention, wherein the chronic wound is selected from the group consisting of a pressure ulcer, a diabetic ulcer on the extremities, a venous leg ulcer, an arterial leg ulcer, a mixed leg ulcer, and a chronic wound associated with Behçet's disease.

A pressure ulcer, also called decubitus ulcer and, popularly, bedsore or pressure sore, within the meaning of the present invention, is defined as an area of unrelieved pressure over a defined area, usually over a bony prominence, resulting in ischemia, cell death, and tissue necrosis (National Pressure Ulcer Advisory Panel (NPUAP)). Pressure ulcers are often caused by conditions such as bedriddeness or boundness to a wheelchair.

Diabetic ulcers on the extremities, in particular diabetic foot ulcers, within the meaning of the present invention, are defined as sores on the feet that occur in 15% of diabetic patients some time during their lifetime. Diabetic foot ulcers occur as a result of various factors, such as mechanical changes in conformation of the bony architecture of the foot, peripheral neuropathy, and atherosclerotic peripheral arterial disease, all of which occur with higher frequency and intensity in the diabetic population.

Venous leg ulcers within the meaning of the present invention, are defined as chronic lower-limb ulcerations resulting from chronic venous insufficiency, leading to a breakdown of the tissue and an ulcer.

Arterial leg ulcer within the meaning of the present invention, are defined as leg ulcers resulting from artery disease, such as atherosclerosis. These ulcers usually affect the toes and feet.

Mixed leg ulcer within the meaning of the present invention, are defined as leg ulcers caused by venous as well as arterial insufficiency or arterial occlusive disease.

Chronic wounds associated with neutrophilic dermatoses within the meaning of this invention include pyoderma gangrenosum.

One embodiment of the present invention is also a compound of the formula (I) as defined above for use in the method for the treatment and/or recurrence rate reduction of a chronic wound according to the invention, wherein the chronic wound is associated with pyoderma gangrenosum.

Pyoderma gangrenosum (PG) is a rare disease, that typically presents as ulcers on the lower extremities but may also occur on any body part. PG may also occur after traumatic events or surgical procedures and ulcers are non-infectious beside a bacterial colonisation. PG is associated in 50 to 70% of the patients with underlying systemic disease, most frequently inflammatory bowel disease (IBD), polyarthritis and hematologic disorders (DeFilippis et al., Br J Dermatol 2015, 172: 1487-1497). Especially the peristomal type of PG is tightly linked to IBD. On the other side only 2% of patients with IBD will develop PG.

One embodiment of the present invention is also a compound of the formula (I) as defined above for use in the method for the treatment and/or recurrence rate reduction of a chronic wound according to the invention, wherein the compound of the formula (I) is administered orally, intravenously, intra-arterially, subcutaneously and/or topically.

One embodiment of the present invention is also a compound of the formula (I) as defined above for use in the method for the treatment and/or recurrence rate reduction of a chronic wound according to the invention, wherein the compound of the formula (I) is administered orally.

One embodiment of the present invention is also a compound of the formula (I) as defined above for use in the method for the treatment and/or recurrence rate reduction of a chronic wound selected from the group consisting of a pressure ulcer, a diabetic ulcer on the extremities, a venous leg ulcer, an arterial leg ulcer, a mixed leg ulcer, and a chronic wound associated with Behçet's disease according to the invention, wherein the compound of formula (I) is administered orally either alone or in addition to one or more physical, biological, topical and/or systemic wound management therapy selected from the group consisting of topical wound dressings, topical antiseptics, wound excision or debridement, weight reduction, appropriate footwear for an offloading effect, PDGF (Regranex), hyperbaric oxygen therapy, compression therapy, wound therapy with negative pressure, maggot debridement therapy, and therapy with systemic antibiotics, wherein the one or more physical, biological, topical and/or systemic wound management therapy is employed simultaneously, sequentially or separately to administering of the compound of formula (I).

Simultaneous employment of physical and/or topical wound management therapies and administration of the compound of formula (I) within the meaning of the present invention is defined as administering the compound of formula (I) while at the same time physical and/or topical wound management therapies are employed.

Sequential employment of physical and/or topical wound management therapies and administration of the compound of formula (I) within the meaning of the present invention is defined as administering the compound of formula (I) and employing physical and/or topical wound management therapies one after the other but in a timely relationship such as one to several hours or days apart.

Separate employment of physical and/or topical wound management therapies and administration of the compound of formula (I) within the meaning of the present invention is defined as administering the compound of formula (I) and employing physical and/or topical wound management therapies in a timely independent manner.

One embodiment of the present invention is also a compound of the formula (I) as defined above for use in the method for the treatment and/or recurrence rate reduction of a chronic wound according to the invention, wherein the compound of formula (I) is applied topically to the wound.

One embodiment of the present invention is also a medicament, comprising a compound of the formula (I) as defined above in combination with an inert, non-toxic, pharmaceutically suitable auxiliary for the treatment and/or recurrence rate reduction of a chronic wound selected from the group consisting of a pressure ulcer, a diabetic ulcer on the extremities, a venous leg ulcer, an arterial leg ulcer, a mixed leg ulcer, and a chronic wound associated with Behçet's disease, wherein the compound of the formula (I) is administered orally and wherein the treatment of the chronic wound causes one or more of the effects selected from increased wound closure rates, shorter time to wound closure, an increase of the reepithelialisation of the chronic wound, an increase of the deposition of extracellular matrix such as collagen in the chronic wound, and a reduction of pain related to the chronic wound.

One embodiment of the present invention is also a medicament, comprising a compound of the formula (I) as defined above in combination with a further active compound selected from the group consisting of lipid metabolism-modulating active compounds, antidiabetics, perfusion-enhancing and/or antithrombotic agents and also antioxidants, aldosterone and mineralocorticoide receptor antagonists, vasopressin receptor antagonists, organic nitrates and NO donors, IP receptor agonists, EP receptor agonists and antagonists, positive inotropic compounds, ACE inhibitors, cGMP- and cAMP-modulating compounds, natriuretic peptides, NO-independent stimulators of guanylate cyclase, NO-independent activators of guanylate cyclase, compounds which inhibit proinflammatory signal transduction cascades, soluble guanylate cyclase (sGC) stimulators or inhibitors, chemokine receptor antagonists, p38 kinase inhibitors, NPY agonists, orexin agonists, anorectics, PAF-AH inhibitors, antiphlogistics, analgesics, AR alpha 2c antagonists, MMP inhibitors, glucocorticoid receptor agonists HIF PH inhibitors, oxidative stress modulators, and pH modulators, systemic or intra/perilesional applied growth factors or systems consisting of living keratinocytes and/or growth factors, topically applied, e.g. as foam or spray, for the treatment and/or recurrence rate reduction of a chronic wound selected from the group consisting of a pressure ulcer, a diabetic ulcer on the extremities, a venous leg ulcer, an arterial leg ulcer, a mixed leg ulcer, and a chronic wound associated with Behçet's disease, wherein the compound of the formula (I) is administered orally and wherein the treatment of the chronic wound causes one or more of the effects selected from increased wound closure rates, shorter time to wound closure, an increase of the reepithelialisation of the chronic wound, an increase of the deposition of extracellular matrix such as collagen in the chronic wound, and a reduction of pain related to the chronic wound.

One embodiment of the present invention is also the use of the medicament in connection with (i.e. pre interventional, during the intervention or post interventional) a surgery or intervention aimed to treat the chronic wound, such as a varicous vein stripping, an arterial baloon dilatation or an aterial bypass surgery, surgical debridement and/or a autologous or heterologous skin transplantation (e.g. by using mesh graft technique) of the chronic wound.

One embodiment of the present invention is also a method for the treatment and/or recurrence rate reduction of a chronic wound selected from the group consisting of a pressure ulcer, a diabetic ulcer on the extremities, a venous leg ulcer, an arterial leg ulcer, a mixed leg ulcer, and a chronic wound associated with Behçet's disease, wherein an effective amount of at least one compound of the formula (I) as defined above or of a medicament as defined above is administered orally or topically to a patient in need thereof and wherein the treatment of the chronic wound causes one or more of the effects selected from increased wound closure rates, shorter time to wound closure, an increase of the reepithelialisation of the chronic wound, an increase of the deposition of extracellular matrix such as collagen in the chronic wound, and a reduction of pain related to the chronic wound.

One embodiment of the present invention is also a compound of the formula (I) as defined above for use in a method for the treatment of neutrophilic dermatoses selected from Behçet's disease, PAPA-syndrome, PASH syndrome, SAPHO syndrome, and subcorneal pustular dermatosis.

One embodiment of the present invention is also a compound of the formula (I) as defined above for use in the method for the treatment of neutrophilic dermatoses selected from Behçet's disease, PAPA-syndrome, PASH syndrome, SAPHO syndrome, and subcorneal pustular dermatosis according to the invention, wherein the compound of the formula (I) is administered orally, intravenously, intra-arterially, subcutaneously and/or topically.

One embodiment of the present invention is also a compound of the formula (I) as defined above for use in the method for the treatment of neutrophilic dermatoses selected from Behçet's disease, PAPA-syndrome, PASH syndrome, SAPHO syndrome, and subcorneal pustular dermatosis according to the invention, wherein the compound of the formula (I) is administered orally.

One embodiment of the present invention is also a compound of the formula (I) as defined above for use in the method for the treatment of autoimmune blistering dermatoses selected from the group consisting of pemphigus, bullous pemphigoid, epidermolysis bullosa acquisita, mucous membrane pemphigoid, pemphigoid gestationis, linear IgA dermatosis, and dermatitis herpetiformis according to the invention, wherein the compound of the formula (I) is administered orally, intravenously, intra-arterially, subcutaneously and/or topically.

One embodiment of the present invention is also a compound of the formula (I) as defined above for use in the method for the treatment of autoimmune blistering dermatoses selected from the group consisting of pemphigus, bullous pemphigoid, epidermolysis bullosa acquisita, mucous membrane pemphigoid, pemphigoid gestationis, linear IgA dermatosis, and dermatitis herpetiformis according to the invention, wherein the compound of the formula (I) is administered orally.

One embodiment of the present invention is also a medicament, comprising a compound of the formula (I) as defined above in combination with an inert, non-toxic, pharmaceutically suitable auxiliary for use in a method for the treatment of neutrophilic dermatoses selected from Behçet's disease, PAPA-syndrome, PASH syndrome, SAPHO syndrome, and subcorneal pustular dermatosis or for use in a method for the treatment of autoimmune blistering dermatoses selected from the group consisting of pemphigus, bullous pemphigoid, epidermolysis bullosa acquisita, mucous membrane pemphigoid, pemphigoid gestationis, linear IgA dermatosis, and dermatitis herpetiformis.

One embodiment of the present invention is also a medicament, comprising a compound of the formula (I) as defined above in combination with a further active compound selected from the group consisting of lipid metabolism-modulating active compounds, antidiabetics, perfusion-enhancing and/or antithrombotic agents and also antioxidants, aldosterone and mineralocorticoide receptor antagonists, vasopressin receptor antagonists, organic nitrates and NO donors, IP receptor agonists, EP receptor agonists and antagonists, positive inotropic compounds, ACE inhibitors, cGMP- and cAMP-modulating compounds, natriuretic peptides, NO-independent stimulators of guanylate cyclase, NO-independent activators of guanylate cyclase, compounds which inhibit proinflammatory signal transduction cascades, soluble guanylate cyclase (sGC) stimulators or inhibitors, chemokine receptor antagonists, p38 kinase inhibitors, NPY agonists, orexin agonists, anorectics, PAF-AH inhibitors, antiphlogistics, analgesics, AR alpha 2c antagonists, MMP inhibitors, glucocorticoid receptor agonists HIF PH inhibitors, oxidative stress modulators, and pH modulators, systemic or intra/perilesional applied growth factors or systems consisting of living keratinocytes and/or growth factors, topically applied, e.g. as foam or spray, for the treatment of neutrophilic dermatoses selected from Behçet's disease, PAPA-syndrome, PASH syndrome, SAPHO syndrome, and subcorneal pustular dermatosis or for use in a method for the treatment of autoimmune blistering dermatoses selected from the group consisting of pemphigus, bullous pemphigoid, epidermolysis bullosa acquisita, mucous membrane pemphigoid, pemphigoid gestationis, linear IgA dermatosis, and dermatitis herpetiformis.

Behçet's disease (also called Adamantiades-Behçet's disease (BD)) is a multisystem disease with oral aphthosis and at least two of the following symptoms: genital aphthae, synovitis, posterior uveitis, cutaneous pustular vasculitis or meningoencephalitis. BD has been reported to occur in association with other neutrophilic dermatoses such as pyoderma gangrenosum and Sweet's syndrome.

PAPA-syndrome (pyogenic arthritis, pyoderma gangrenosum, acne), is a rare autosomal/dominant autoinflammatory condition caused by aberrant production of interleukin 1 (Demidowich, A. P., et al, Arthritis Rheum (2012) 64(6): 2022-2027). It is associated with a mutation of PSTPIP1 gene, also known as CD2 antigen-binding protein 1 (Demidowich et al., Arthritis & Rheumatism 2012, 64: 2022-2027). PSTPIP1 encodes a proline-serine-threonine phosphatase-interacting protein that binds to pyrin, which regulates inflammasomes (DeFilippis et al., Br J Dermatol 2015, 172: 1487-1497).

Neutrophil infiltration and thus high loads of neutrophil elastase are hallmarks of PASH syndrome (pyoderma gangrenosum, acne, and suppurative hidradenitis) and variations of the PAPA syndrome. Braun-Falco and colleagues described two patients with pyoderma gangrenosum, acne, and suppurative hidradenitis but without pyogenic arthritis. The syndrome was named PASH syndrome (Braun-Falco et al. 2012, J Am Acad Dermatol 66(3): 409-415). They found an increased number of CCTG microsatellite repeats in the PSTPIP1 promoter region. Since PAPA and PASH share the same downstream pathomechanism, clinical presentation of pyoderma gangrenosum and acne-like lesions and histopathological findings of dense neutrophil infiltrates are comparable between both diseases.

SAPHO syndrome (Synovitis, ‘acne,’ pustulosis, ‘hyperostosis,’ osteomyelitis) Since the 1960s diseases with associations of pustular dermatoses and osteoarthritis have been described. In 1987 Chamot et al. suggested the term SAPHO-syndrome as acronym for synovitis, acne, pustulosis, hyperostosis, osteitis (Chamot et al., 1987, Rev Rhum Mal Osteoartic 54(3): 187-196). While the pathogenic mechanisms in contrast to PAPA and PASH in SAPHO-syndrome still remains unclear, HLA-B27-associations have been described (Rukavina 2015, J Child Orthop. 2015, 9:19-27).

Subcorneal pustular dermatosis (SCPD) was first described in 1956 by Sneddon and Wilkinson (Sneddon and Wilkinson 1956, Br J Dermatol 68(12): 385-394). Usually SCPD starts in the folds and rapidly within one to two days spreads over the whole body. The clinical sign are pustules usually on normal appearing skin—less frequently on erythematous skin. Lesions may be accompanied by pain but they usually don't itch (Sneddon and Wilkinson 1956, Br J Dermatol 68(12): 385-394, and Sneddon and Wilkinson 1979, Br J Dermatol 100(1):61-68.).

One embodiment of the present invention is also a compound of the formula (I) as defined above for use in a method for the treatment of autoimmune blistering dermatoses (AIBDs) selected from the group consisting of pemphigus, bullous pemphigoid (BP), epidermolysis bullosa acquisita (EBA), mucous membrane pemphigoid (MMP), pemphigoid gestationis (PG), linear IgA dermatosis, and dermatitis herpetiformis (DH).

Bullous pemphigoid is an autoimmune subepidermal blistering skin diseases associated with IgG autoantibodies against the dermal-epidermal junction, wherein the autoantibodies are targeted against hemidesmosomal antigens BP180 and BP230. Epidermolysis bullosa acquisita is also an autoimmune subepidermal blistering skin diseases associated with IgG autoantibodies against the dermal-epidermal junction. In this disease, the autoantibodis target type VII collagen (Shimanovich et al., J Pathol 2004; 204:619-527).

Pemphigus including all his sub-entitites is an autoimmune intradermal blistering skin disease characterized by autoantibodies against intraepidermal demosomal structure proteins. Pemphigus is a chronic disease with a sometimes severe clinical picture, relapses, and prolonged immunosuppressive treatment that impairs both physical and psychosocial aspects of quality of life. To the group of Pemphigus diseases belong the following specific entities: subcorneal pustular dermatosis, pemphigus vulgaris, pemphigus vegetans, pemphigus foliaceus, pemphigus erythematosus, endemic pemphigus, Northern Colombia Pemphigus herpetiformis, paraneoplastic pemphigus, drug-induced pemphigus and the IgA pemphigus. Autoantibodies to intradermal target antigens causes loss of cell-cell adhesion between keratinocytes and intraepithelial blister formation called acantholysis. There is evidence that occult underlying systemic disease such as Systemic Lupus, hematological malignancies or IBD may cause those symptoms by their common pathophysiology.

Epidermolysis bullosa acquisita (EBA) is a rare subepidermal blistering disease, characterized by chronic course, resistance to therapy, and often debilitating sequelae. It is mediated by autoantibodies against type VII collagen of the BMZ in stratified squamous epithelia. Recently, type VII collagen was also found in the BMZ of the colon and in the intestinal epithelium.

Mucous membrane pemphigoid (MMP), previously known as cicatricial pemphigoid, is a rare but well-defined variant of pemphigoid, characterized by erosive, scarring, subepidermal blistering lesions of mucosal surfaces, particularly of the oral and ocular mucosa. Pemphigoid gestationis (PG), previously known as “herpes gestationis,” is a rare pregnancy-specific form of pemphigoid. Linear IgA dermatosis, is a rare chronic autoimmune bullous disease associated with IgA anti-BMZ antibodies. Dermatitis herpetiformis (DH), also known as Duhring's disease, is an uncommon subepidermal blistering disease characterized by an intensely pruritic cutaneous eruption associated with a gluten-sensitive enteropathy.

Although the primary problem in AIBDs is in the skin and/or mucous membranes, they are associated with secondary systemic complications that may be potentially fatal. Immunobullous diseases provide another challenge, because their treatment warrants the use of high doses of systemic corticosteroids and immunosuppressive drugs associated with various adverse side effects and high risk for serious systemic complications.

All AIBDs are difficult to treat and since no causal therapy is available, often patients are resistant to all conventional therapies. Systemic GSc in combination with immunosuppressive agents, including immunoablative cyclophosphamide, cyclosporine A, plasmapheresis, immunoapheresis, rituximab, and, most recently, alemtuzwnab are recommended in severe cases. Selective inhibition of human leucocyte elastase (or gelatinase B/MMP-9) was also found to result in suppression of blistering. These findings strongly suggest that elastase and gelatinase B are essential for granulocyte-mediated proteolysis resulting in dermal-epidermal separation in EBA, Pemphigus or BP patients' skin (Vassileva, S. et al., Clinics in Dermatology 2014, 32: 364-375; Shimanovic, I. et al., J Pathol 2004, 204: 519-527).

The compounds according to the invention have an unforeseeable useful spectrum of pharmacological activity, including useful pharmacokinetic properties. As specific neutrophil elastase inhibitors, the compounds of formula (I) for use in the method of the invention modulate protease activity in the wound environment and offer a new, and the first oral innovative therapeutic approach for chronic wounds.

The compounds according to the invention can be used alone or, if required, in combination with a companion diagnostic test also as a protease or elastase bed side point of care test or a lab based method to identify the elastase or protease status or in combination with other active compounds. The present invention further provides medicaments comprising a compound according to the invention and one or more further active compounds, in particular for treatment and/or prophylaxis of the disorders mentioned above. Suitable active ingredients for combination are, by way of example and by way of preference:
lipid metabolism-modulating active ingredients, by way of example and by way of preference from the group of the HMG-CoA reductase inhibitors from the class of the statins such as, by way of example and by way of preference, lovastatin, simvastatin, pravastatin, fluvastatin, atorvastatin, rosuvastatin, cerivastatin or pitavastatin, inhibitors of HMG-CoA reductase expression, squalene synthesis inhibitors such as, by way of example and by way of preference, BMS-188494 or TAK-475, ACAT inhibitors such as, by way of example and by way of preference, melinamide, pactimibe, eflucimibe or SMP-797, LDL receptor inductors, cholesterol absorption inhibitors such as, by way of example and by way of preference, ezetimibe, tiqueside or pamaqueside, polymeric bile acid adsorbers such as, by way of example and by way of preference, cholestyramine, colestipol, colesolvam, CholestaGel or colestimide, bile acid reabsorption inhibitors such as, by way of example and by way of preference, ASBT (=IBAT) inhibitors such as elobixibat (AZD-7806), S-8921, AK-105, canosimibe (BARI-1741, AVE-5530), SC-435 or SC-635, MTP inhibitors such as, by way of example and by way of preference, implitapide or JTT-130, lipase inhibitors such as, by way of example and by way of preference, orlistat, LpL activators, fibrates, niacin, CETP inhibitors such as, by way of example and by way of preference, torcetrapib, dalcetrapib (JTT-705) or CETP vaccine (Avant), PPAR-γ and/or PPAR-δ agonists such as, by way of example and by way of preference, pioglitazone or rosiglitazone and/or endurobol (GW-501516), RXR modulators, FXR modulators, LXR modulators, thyroid hormones and/or thyroid mimetics such as, by way of example and by way of preference, D-thyroxine or 3,5,3′-triiodothyronine (T3), ATP citrate lyase inhibitors, Lp(a) antagonists, cannabinoid receptor 1-antagonists such as, by way of example and by way of preference, rimonabant or surinabant (SR-147778), leptin receptor agonists, bombesin receptor agonists, histamine receptor agonists, agonists of the niacin receptor such as, by way of example and by way of preference, niacin, acipimox, acifran or radecol, and the antioxidants/radical scavengers such as, by way of example and by way of preference, probucol, succinobucol (AGI-1067), BO-653 or AEOL-10150;
antidiabetics mentioned in Die Rote Liste 2014, chapter 12. Antidiabetics are preferably understood as meaning insulin and insulin derivatives and also orally effective hypoglycemically active compounds. Here, insulin and insulin derivatives include both insulins of animal, human or biotechnological origin and mixtures thereof. The orally effective hypoglycaemically active compounds preferably include sulphonylureas, biguanides, meglitinide derivatives, glucosidase inhibitors and PPAR-gamma agonists. Sulfonylureas which may be mentioned are, by way of example and by way of preference, tolbutamide, glibenclamide, glimepiride, glipizide or gliclazide, biguanides which may be mentioned are, by way of example and by way of preference, metformin, meglitinide derivatives which may be mentioned are, by way of example and by way of preference, repaglinide or nateglinide, glucosidase inhibitors which may be mentioned are, by way of example and by way of preference, miglitol or acarbose, oxadiazolidinones, thiazolidinediones, GLP 1 receptor agonists, glucagon antagonists, insulin sensitizers, CCK 1 receptor agonists, leptin receptor agonists, inhibitors of liver enzymes involved in the stimulation of gluconeogenesis and/or glycogenolysis, modulators of glucose uptake and potassium channel openers such as, for example, those disclosed in WO 97/26265 and WO 99/03861;
hypotensive active compounds, by way of example and by way of preference from the group of the calcium antagonists such as, by way of example and by way of preference, nifedipine, amlodipine, verapamil or diltiazem, angiotensin AII antagonists such as, by way of example and by way of preference, losartan, valsartan, candesartan, embusartan or telmisartan, ACE inhibitors such as, by way of example and by way of preference, enalapril, captopril, ramipril, delapril, fosinopril, quinopril, perindopril or trandopril, beta receptor blockers such as, by way of example and by way of preference, propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol, alpha receptor blockers such as, by way of example and by way of preference, prazosin, ECE inhibitors, rho-kinase inhibitors and of the vasopeptidase inhibitors, and also of the diuretics such as, by way of example and by way of preference, a loop diuretic such as furosemide, bumetanide or torsemide, or a thiazide or thiazide-like diuretic such as chlorothiazide or hydrochlorothiazide or A1 antagonists such as rolofylline, tonopofylline and SLV-320;
agents which lower the symphathetic tone such as, by way of example and by way of preference, reserpin, clonidine or alpha-methyldopa, or in combination with a potassium channel agonist such as, by way of example and by way of preference, minoxidil, diazoxide, dihydralazine or hydralazine;
agents with antithrombotic action such as, by way of example and by way of preference, from the group of the platelet aggregation inhibitors such as, by way of example and by way of preference, aspirin, clopidogrel, ticlopidine, cilostazol or dipyridamole, or of the anticoagulants such as thrombin inhibitors such as, by way of example and by way of preference, ximelagatran, melagatran, bivalirudin or clexane, a GPIIb/IIIa antagonist such as, by way of example and by way of preference, tirofiban or abciximab, a factor Xa inhibitor such as, by way of example and by way of preference, rivaroxaban, edoxaban (DU-176b), apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428, with heparin or a low molecular weight (LMW) heparin derivative or with a vitamin K antagonist such as, by way of example and by way of preference, coumarin;
aldosterone and mineralocorticoid receptor antagonists such as, by way of example and by way of preference, spironolactone, eplerenone or finerenone;
vasopressin receptor antagonists such as, by way of example and by way of preference, conivaptan, tolvaptan, lixivaptan or satavaptan (SR-121463);
organic nitrates and NO donors such as, by way of example and by way of preference, sodium nitroprusside, nitroglycerol, isosorbide mononitrate, isosorbide dinitrate, molsidomine or SIN-1, or in combination with inhalative NO;
IP receptor agonists, preferred examples being iloprost, treprostinil, beraprost and selexipag (NS-304);
Prostaglandin EP receptor agonists and antagonists
compounds having a positive inotropic effect, preferred examples being cardiac glycosides (digoxin), beta-adrenergic and dopaminergic agonists such as isoproterenol, adrenaline, noradrenaline, dopamine and dobutamine;
calcium sensitizers, a preferred example being levosimendan;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, especially PDE 5 inhibitors such as sildenafil, vardenafil and tadalafil, and PDE 3 inhibitors such as milrinone;
natriuretic peptides, for example atrial natriuretic peptide (ANP, anaritide), B-type natriuretic peptide or brain natriuretic peptide (BNP, nesiritide), C-type natriuretic peptide (CNP) and urodilatin;
NO-independent but haem-dependent stimulators of guanylate cyclase, such as especially the compounds described in WO 00/06568, WO 00/06569, WO 02/42301 and WO 03/095451;
NO- and haem-independent activators of guanylate cyclase, such as especially the compounds described in WO 01/19355, WO 01/19776, WO 01/19778, WO 01/19780, WO 02/070462 and WO 02/070510;
compounds which inhibit proinflammatory signal transduction cascades, for example tyrosine kinase inhibitors and multikinase inhibitors, especially sorafenib, imatinib, gefitinib and erlotinib; and/or
compounds which inhibit proinflammatory signal transduction cascades, for example NFkappaB or AP1; and/or
compounds which influence the energy metabolism of the heart, such as, for example, etomoxir, dichloroacetate, ranolazine and trimetazidine.
chemokine receptor antagonists such as maraviroc,
p38 kinase inhibitors,
NPY agonists,
orexin agonists,
anorectics,
PAF SAH inhibitors,
antiphlogistics,
analgesics,
antidepressives and other psychopharmaceuticals,
selective AR alpha 2c antagonists, such as, for example, compounds known from WO2015091414, such as [4-(3,4-dihydroisoquinolin-2(1H)-yl)piperidin-1-yl][2-(2-oxa-6-azaspiro[3.3]hept-6-yl)pyrimidin-5-yl]methanone,
MMP inhibitors,
selective glucocorticoid receptor agonists (SEGRA), such as, for example 5-{[1-(2-Chloro-3-fluoro-4-methoxyphenyl)-3,3.3-trifluoro-2-hydroxy-2-(methoxymethyl)propyl]amino}-7-fluoro-1H-quinolin-2-one known from WO2009/065503.
HIF PH inhibitors,
oxidative stress modulators,
pH modulators such as pH modulating ointments or wound covers.

In the context of the present invention, particular preference is given to combinations comprising at least one of the compounds according to the invention and one or more further active compounds selected from the group consisting of HMG-CoA reductase inhibitors (statins), diuretics, beta-receptor blockers, organic nitrates and NO donors, ACE inhibitors, angiotensin AII antagonists, aldosterone and mineralocorticoid receptor antagonists, vasopressin receptor antagonists, platelet aggregation inhibitors and anticoagulants, and also to their use for the treatment and/or recurrence rate reduction of a chronic wound selected from the group consisting of a pressure ulcer, a diabetic ulcer on the extremities, a venous leg ulcer, an arterial leg ulcer, a mixed leg ulcer, and a chronic wound associated with Behçet's disease, wherein the compound of the formula (I) is administered orally and wherein the treatment of the chronic wound causes one or more of the effects selected from increased wound closure rates, shorter time to wound closure, an increase of the reepithelialisation of the chronic wound, an increase of the deposition of extracellular matrix such as collagen in the chronic wound, and a reduction of pain related to the chronic wound.

Particular preference in the context of the present invention is given to combinations comprising at least one of the compounds according to the invention and one or more further active compounds selected from the group consisting of heparin, antidiabetics, ACE inhibitors, diuretics and antibiotics, and also to their use for the treatment and/or recurrence rate reduction of a chronic wound, wherein the treatment of the chronic wound selected from the group consisting of a pressure ulcer, a diabetic ulcer on the extremities, a venous leg ulcer, an arterial leg ulcer, a mixed leg ulcer, and a chronic wound associated with Behçet's disease, wherein the at least one of the compounds according to the invention is administered orally, which causes one or more of the effects selected from increased wound closure rates, shorter time to wound closure, an increase of the reepithelialisation of the chronic wound, an increase of the deposition of extracellular matrix such as collagen in the chronic wound, and a reduction of pain related to the chronic wound.

The compounds according to the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds according to the invention can be administered in suitable administration forms for these administration routes.

Suitable administration forms for oral administration are those which function according to the prior art and deliver the compounds according to the invention rapidly and/or in modified fashion, and which contain the compounds according to the invention in crystalline and/or amorphized and/or dissolved form, for example tablets (uncoated or coated tablets, for example having enteric coatings or coatings which are insoluble or dissolve with a delay and control the release of the inventive compound), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can be accomplished with avoidance of an absorption step (for example by an intravenous, intraarterial, intracardiac, intraspinal or intralumbar route) or with inclusion of an absorption (for example by an intramuscular, subcutaneous, intracutaneous, percutaneous or intraperitoneal route). Suitable administration forms for parenteral administration include injection and infusion formulations in the form of solutions, suspensions, emulsions, lyophilizates or sterile powders.

Oral administration is preferred.

In the exemplary use of the compounds of the formula (I) for the treatment and/or recurrence rate reduction of chronic wounds, preference, in addition to oral administration, is also given to administration in the form of a topical formulation.

For the other administration routes, suitable examples are inhalation medicaments (including powder inhalers, nebulizers), nasal drops, solutions or sprays; tablets for lingual, sublingual or buccal administration, films/wafers or capsules, suppositories, ear or eye preparations, vaginal capsules, aqueous suspensions (lotions, shaking mixtures), lipophilic suspensions, ointments, creams, transdermal therapeutic systems (for example patches), milk, pastes, foams, dusting powders, implants or stents.

The compounds according to the invention can be converted to the administration forms mentioned. This can be accomplished in a manner known per se by mixing with inert, non-toxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctants.

The present invention further provides medicaments comprising at least one inventive compound, preferably together with one or more inert nontoxic pharmaceutically suitable excipients, and the use thereof for the purposes mentioned above.

According to a further embodiment, it has proved advantageous, in the case of oral or parenteral administration, to administer amounts in a range of 0.1 to 100 or from 0.5 to 50, or from 0.5 to 10 or from 0.5 to 5, or from 0.7 to 5 or from 0.7 to 3 or from 0.7 to 2 or from 1 to 2 mg/day to achieve effective results.

Nevertheless, it may optionally be necessary to deviate from the stated amounts, namely depending on body weight, route of administration, individual response to the active substance, type of preparation and time point or interval when application takes place. Thus, in some cases it may be sufficient to use less than the aforementioned minimum amount, whereas in other cases the stated upper limit must be exceeded. When applying larger amounts, it may be advisable to distribute these in several individual doses throughout the day.

According to a further embodiment, the compounds of formula (I) according to the invention are administered orally once or twice or three times a day. According to a further embodiment, the compounds of formula (I) according to the invention are administered orally once or twice a day. According to a further embodiment, the compounds of formula (I) according to the invention are administered orally once a day. For the oral administration, a rapid release or a modified release dosage form may be used.

According to a further embodiment, the compounds of formula (I) according to the invention are administered orally once or twice daily in a chronic way until the wound has healed or has significantly improved and can be controlled without the use of the compound. Alternative to the oral administration is i.v. administration or a topical treatment, e.g. as spray, gel, foam, ointment or similar or as active ingredient of a wound dressing, or wound cover or other wound treatment concept.

The percentages in the following tests and examples are percentages by weight, unless stated otherwise; parts are parts by weight. Proportions of solvents, dilution ratios and concentrations for liquid/liquid solutions refer in each case to the volume or interval over which administration takes place.

### A) ASSESSMENT OF PHYSIOLOGICAL EFFICACY

The following abbreviations are used:

| | |
|---|---|
| AC - POX | (sodium) acetate citrate buffer for myeloperoxidase |
| buffer | activity assay |
| Brij | polyoxyethylene lauryl ether |
| BW | body weight |
| DEPO | DMSO-Ethanol-Peanut Oil |
| DMSO | Dimethylsulfoxid |
| ECM | extracellular matrix |
| HaCaT | Human adult low Calcium high Temperature keratinocytes |
| HYP | Hydroxyproline |
| MPO | Myeloperoxidase |
| MWP | Microwellplate |
| NE | Neutrophil Elastase |
| o.d. | once daily |
| PEG | Poly Ethylene Glycol |
| PO | Peanut Oil |
| RT | room temperature |
| TMB | 3,3′,5,5′-Tetramethylbenzidin |

The suitability of the compounds according to the invention for treating chronic wounds was demonstrated in the following assays:

B-1) In Vitro Assays

B-1a) HaCaT Modified Scratch Assay

The aim is to test whether Neutrophil Elastase (NE) inhibitors can effectively improve Elastase-induced healing delay in an in vitro model of epithelial healing (modified scratch assay on HaCaT cells).

The modified scratch-wound assay is a simple, reproducible assay commonly used to measure basic cell migration parameters such as speed, persistence, and polarity. Cells are grown to confluence and a thin “wound” is introduced by scratching with a pipette tip. In the modified version, a plastic plug is placed into the tissue well while the cells adhere and grow to confluence. When the plug is pulled, a circular “wound” emerges. Cells at the wound edge polarise and migrate into the wound space. Advantages of this assay are that it does not require the use of specific chemoattractants or gradient chambers and it generates a strong directional migratory response. At the given time points, the size of the free area is measured.

Method:

Oris Cell Migration Assay (collagen I coated) with HaCaT cells. Establishing a dose response curve for test compounds with and without addition of 7.5 μg/mL human neutrophil elastase (hNE).

For harvesting the cells, the medium was first extracted from the cell culture flask, then washed with PBS and 10-20 ml aspirated again. 2.5 ml (75 cm² flask bottom area) or 5 ml (162 cm²) TrypLE Express were added, as well as the same amount of PBS followed by incubating the cells for 10 minutes (37° C.). The cells were transferred into a tube, centrifuged (250×g, 10 min, RT=room temperature) and resuspended in medium.

The following cell concentration was used: 7×10⁵ cells/ml=7×10⁴ cells/well/100 μl.

On day 0, the selected cells were seeded into the wells and incubated for 6 hours (37° C., 90-95% rel. humidity, 5% CO₂). In this time, the cells adhered and grew to confluence. The compounds were then added to achieve the below indicated concentrations. The plugs were then removed.

Once all components were pipetted to the cells, one photo per well was taken including the “wound” (0 h). A Zeiss AxioObserver (5× lens, bright field, 5.5 V light exposure ˜2.2 ms) and the AxioVision software was used. After creating the pictures, the microplate was incubated in an incubator (37° C., 90-95% rel. humidity, 5% CO₂). 16 h later pictures were taken again.

The relative healing was calculated, i.e. the percentage of closed wound area compared to baseline at 0 h.

Wound area after 0 hours=0% healing

Wound area of 0 μm²=100% healing

Formulae for the calculation of wound healing:

Wound area after 0 hours=average wound area of untreated wells after 0 h hours

Wound healing %=100−(‘wound surface after x hours’*100/‘wound surface after 0 hours’)

From the calculated relative wound healing a graphic in GrapPad prism was created, and EC₅₀ values were determined.

Materials:

Human Keratinocytes (HaCaT) Cell Line Service #300493

Culture media: RPMI-1640+Glutamax-I, 500 ml, stored at 4° C. (Invitrogen #61870)+1% FBS, 5 ml, stored at −20° C. (Invitrogen #10500)+50 U/ml penicillin and 50 ug/ml streptomycin, 2.5 ml, stored at −20° C. (Invitrogen #15140)

Dulbecco's PBS w/o Ca₂+ and Mg₂+(Invitrogen #14190)

TrypLE Express, stored at +4° C. (Invitrogen #12604)

Casy® cell counter system

Trypan Blue Solution (Fluka #93595)

96 well microtiter plate (MTP), round-bottomed (Corning #3799)

MediumD=medium+0.4% DMSO

DMSO (dimethyl sulfoxide) (Riedel-de Haën #60153)

Oris Cell Migration Assay (Platypus # CMACC5.101)

Evaluation

For evaluation of pictures the software ImageJ was used. For this, the ZVI files were exported within the AxioVision software as jpg. This jpg files were then opened by ImageJ. Using the Tools panel (Mode: 4-connected/Tolerance=5-20) the to be measured surface area was marked. Pixel scaling setting: 488 pixels=1000 μm.

Results

Supplementation of the cell culture media with 7.5 μg/mL human neutrophil elastase (i.e. concentrations as present also in exudates from chronic wounds) completely abrogates the migration of keratinocytes into the artifical wound. This reepithelialization-impairing effect can be almost entirely, potently and concentration-dependently be corrected by adding Example 1, Example 3 or Example 5.

TABLE 1

| | Example 1 | Example 3 | Example 5 |
|---|---|---|---|
| Bottom | 89.76 | 90.41 | 88.88 |
| Top | 41.13 | 42.75 | 38.86 |
| IC₅₀ [M] | 3.87 × 10⁻⁸ | 5.74 × 10⁻⁸ | 2.71 × 10⁻⁸ |
| Span | −48.62 | −47.65 | −50.02 |
| Degrees of Freedom | 9 | 9 | 9 |
| R square | 0.97 | 0.97 | 0.95 |
| Number of points Analyzed | 12 | 12 | 12 |

Conclusion: All NE inhibitors tested (and Example 1, Example 5, and Example 3) were potently and effectivly able to block detrimental effects of NE in this assay.

B-2) In Vivo Assays

B-2a) Improved Reepithelialization and Visual Healing in Wounds of Tight Skin Mice (TSK)

The Tight Skin 2 (Tsk2) mouse model of systemic sclerosis (SSc) has many features of human disease, including tight skin, excessive collagen deposition, alterations in the extracellular matrix (ECM), increased elastic fibers, and occurrence of antinuclear antibodies with age. A tight skin phenotype is observed by 2 weeks of age, but measurable skin fibrosis is only apparent at 10 weeks. However, it is known that the healing of skin wounds is delayed in Tsk mice. Both deregulation of ECM deposition as well as neutrophil dependent inflammatory processes may contribute to this phenotype.

OBJECTIVE: To evaluate wound closure/reepithelialization promoting effects of the Neutrophil Elastase Inhibitor Example 3 in a fibrosis-related model of delayed wound healing (Tight Skin Mouse, TSK). METHODS: Animals: mice, male+female, strain: B6.Cg-FBN1 <Tsk>−/−(wt) and +/−(het), age: 5 weeks upon delivery, breeder: Bayer. Food (ssniff R/M-H) and water was provided ad libitum. Experimental procedure: Mice were randomized at day 0. At day 0, 3 full thickness excision wounds were induced. For that, animals were anesthetized (Rompun Ketavet/NaCl 0.9% [1+3+16]) in 150 μl/25 g BW. The lower back was shaved, then moistened with tap water. Pilca depilation cream was applied and left for 3-5 minutes to act, then wiped off. To prevent the animals from cooling down during the procedure, they were placed on warming mats. Skin was excised using 6 mm diameter round scalpels. After wake up mice were distributed to individual cages. Treatment: Day 0 to 11 twice daily per os. Example 3 was dissolved in PEG400 as a vehicle. Administration was twice daily (bid). Skin samples were harvested into formalin (3.7% in PBS) at day 12 after sacrifying with isoflurane/O₂/N₂O anesthesia followed by dislocation of the cervical spine. After soaking in formalin (24-72 h), the samples were transferred to paraffin and then cut. Staining of cytokeratin 16 was performed using rabbit polyclonal anti-Keratin 16 (CK16, KRT16, ABIN265495), 1 mg/ml anti-CK16 antibody by IHC-Peroxidase (in a 1:100 dilution) EnVision+ System-HRP (DAB), for Rabbit Primary Antibodies (Dako# K4011).

RESULTS: Example 3 had significant visual wound healing improving effects regarding wound area reduction over time (FIG. 1). Effects were most prominent on d7 and d9. All dosages were similarly effective, i.e. a clear dose response ratio remained to be established. Visual findings (wound size measurement) were confirmed histologically, i.e. reepithelialization was significantly further advanced in Example 3-treated groups vs. vehicle controls (Table 3). Based on the size of the induced wounds, 6000 μm was set as 100%. The epidermal gap, i.e. the distance between the epidermal edges was measured by microscopic histometry. The width of the gap was subtracted from the initial wound. The delta represented the reepithelialized part of the wound and was expressed as reepithelialization in % of the initial wound diameter, i.e. 100% equaled complete reepithelialization or full wound closure.

CONCLUSION: Example 3 induced accelerated wound healing in a model of delayed wound healing (TSK mouse).

TABLE 2

| Animals (n) | Species | Sex | Strain | Origin |
|---|---|---|---|---|
| 67 | Mouse | m. and f. | B6.Cg- | Bayer |
| | | | Fbn1<Tsk>−/− | |
| | | | (wt) and +/− | |
| | | | (het) | |

| | | | | Dose [mg/kg | | |
|---|---|---|---|---|---|---|
| Group | n/group | | Example | BW] | Vehicle | Regimen |
| 1 | 12 | wt | — | — | — | — |
| 2 | 11 | TSK | — | — | PO PEG400 | p.o., bid |
| 3 | 11 | TSK | Example 3 | 2.5 | PO PEG400 | p.o., bid |
| 4 | 11 | TSK | Example 3 | 5 | PO PEG400 | p.o., bid |
| 5 | 11 | TSK | Example 3 | 10 | PO PEG400 | p.o., bid |
| 6 | 11 | TSK | Example 3 | 20 | PO PEG400 | p.o., bid |

| | | | |
|---|---|---|---|
| Vehicle: | PO, PEG400 | PO, PEG400 (5%) | po. 5 ml/kg BW p.o. |

TABLE 3
Reepithelialization of foil thickness skin excision wounds in
Tsk mice at d 12 of healing as assessed by CK16 immunohistochemical
staining (all wounds including those with eschar).

Wound closure (reepithelialization)
in % of initial epidermal gap
(number of animals)

| | Wild type mouse | TSK mouse |
|---|---|---|
| Vehicle | 100 (n = 7) | 71.9 (n = 8) |
| Example 3; 2.5 mg/kg BW, | n.d. | 95.8 (n = 7) |
| twice daily | | |
| Example 3; 5 mg/kg BW, | n.d. | 93.9 (n = 10) |
| twice daily | | |
| Example 3; 10 mg/kg BW, | n.d. | 99.5 (n = 8) |
| twice daily | | |
| Example 3; 20 mg/kg BW, | n.d. | 100 (n = 10) |
| twice daily | | |

B-2b) Exposure of Example 5 in Plasma and Wounds of Diabetic Mouse (Db/Db), Effects on NE Activity and Wound Size Reduction

Effects of Example 5 on delayed dermal wound healing in insulin-resistant diabetic mice (BKS.Cg-Dock7m+/+Leprdb/J).

Animals

For the experiment n=64 male BKS.Cg−Dock7<m>+/+Lepr<db>/J mice (Charles River Italy) carrying a spontaneous mutation in both alleles of the leptin receptor were used to model delayed wound healing. The db/db mice had an increased blood-sugar level (>300 mg dL⁻¹) compared to the wild-type mice (Blood Sugar: <200 mg dL⁻¹).

All animals were 7-8 weeks old upon delivery and 9 weeks old at the start of the experiment. Animals were kept in single cages with a 12 h light/dark cycle and given chow and sterile drinking water ad libidum. All experiments were performed in accordance with company, regional and federal guidelines for the use of laboratory animals. They were approved and realized in compliance with policies and directives of the LAGESO (Landesamt für Gesundheit and Soziales, Berlin) and all efforts were made to minimize suffering.

Wound Healing

Mice were randomly split into different groups depending on the treatment (db/db only). Starting on the day of wounding, mice were treated daily p.o. with an application volume of 5 mL kg⁻¹ and a dose of 10 mg kg in case of the treated group. Placebo groups received the vehicle of the substance: DMSO+Ethanol+Peanut oil (5+3+92 parts). Treatment with the substance or placebo was always done 3-5 hours before wounding or before mice were sacrificed.

TABLE 4

| Group | Mice/group (n) | Example | Dose [mg/kg BW] |
|---|---|---|---|
| 1 | 10 | — | — |
| 2 | 10 | Example 5 | 0.03 |
| 3 | 10 | Example 5 | 0.1 |
| 4 | 10 | Example 5 | 0.3 |
| 5 | 10 | Example 5 | 1 |
| 6 | 10 | Example 5 | 3 |
| 7 | 10 | Example 5 | 10 |

Vehicle: DMSO/EtOH/Peanutoil, 5/3/92 (v/v/v) 5 ml/kg BW p.o., o.d.

The wounding was conducted in the following manner:
1) db/db mice were anaesthetized with Isoflurane/O₂/N₂O (2,5-3,5%/800 ml/min/500 ml/min), and placed on a heating pad upon induction after application of eye ointment
2) mice were shaven and Pilca was applied for 3-5 minutes to remove the fur and fine hairs
3) two dorsal full thickness wounds were placed with a 8 mm punch biopsie tool after wiping the skin with ethanol and wound size was measured immediately
4) mice were treated with Temgesic and kept on warming pad until recovery from anaesthesia

Wound Size Measurement: Wound size was measured with a sliding caliper. Animals were anaesthetized with 3.5% Isoflurane (O₂=800 mL h⁻¹, N₂O=550 mL h⁻¹) and arranged so that the back and legs were in a flat and relaxed position. Assuming an elliptic shape, wound area was calculated with:

$$A = \frac{π vh}{4} [ {mm}^{2} ]$$

Wound size reduction on day i was calculated from the initial area on day 0 post wounding:

$$Reduction = \frac{{A}_{0} - {A}_{i}}{{A}_{0}} * 100 \% [ \% ]$$

On day 8 post wounding the mice were sacrificed by Isoflurane inhalation. Wounds and surrounding skin were removed after wound size measurement with a 10 mm punch biopsy tool. Wound tissues were weighed, wound 1 was placed in liquid N₂ for myeloperoxidase (MPO) activity measurement; wound 2 was placed in liquid N₂ for NE-activity measurement and wound 3 was placed in formalin (3.7%) for the histological analyses.

Upon sacrification on day 8, 20 h after the last treatment, arterial blood samples were collected into EDTA citrate serum sample tubes for LC/MSMS based quantification of compound exposure.

Tissue Lysis

Frozen wound tissue was placed in the automated homogenizer and 1.5 mL homogenate buffer was added at RT (room temperature). Tissue was then homogenized with the blender for 20 seconds at the highest speed. Homogenate was placed in an ultracentrifuge and separated at 15,000 rpm, 12° C. for 20 min. The supernatant was removed completely and split into aliquots for subsequent NE and MPO analysis.

Neutrophil Elastase Assay

Activity of NE was quantified by monitoring protease activity with a fluorescence labelled substrate (MeOSuc-AAPV-AMC), which is highly specific for NE over other serine proteases such as Proteinase 3 (Castillo et al., Analytical Biochem 1979, 99: 53-64; Wiesner et al., FEBS Lett 2005, 579: 5305-5312). Recombinant murine NE prepared in homogenate buffer was used as a standard curve and homogenate buffer as a blank. For the assay 25 μL of undiluted homogenate/standard/blank were pipetted into a black flat bottom MWP (multi well plate) at RT and mixed with 25 μl of 1 mM MeOSuc-AAPV-AMC prepared in cold TrisBSA buffer. MWP was immediately placed in pre heated plate reader and fluorescence was monitored for 10 min every 30 min at 37° C. and λ_{Ex}=380 nm and λ_{Em}=460 nm. Between measurements, substrate solution was stored in the dark at 4° C. Each sample was analyzed once per plate, but all plates were run as technical replicates with n=4. The plate reader software SoftmaxPro 6.4 was used to calculate V₀ (Initial Velocity) and interpolated against standard curve to calculate the amount of NE after subtracting the blank. The mean result of this determination was used for subsequent analysis.

Myeloperoxidase (MPO) Assay

MPO determination is based on the oxidation of H₂O₂ by a peroxidase in the presence of TMB (3,3′5,5′-Tetramethylbenzidin). The results were quantified with a human MPO standard curve prepared in homogenate buffer, which also serves as a blank. Samples were diluted fifteen fold in homogenate buffer. Of each sample, standard and blank 20 μL were dispensed in a clear flat bottom MWP at RT. Following that 100 μL of fresh 0.2 mM TMB in AC-PDX buffer are added and the reaction was started by adding 12.5 μL of 1 mM H₂O₂ in AC-PDX buffer. After 5-10 min 22.5 μL of 1 N H₂SO₄ was added to stop the reaction and plates were vortexed briefly to evenly distribute the newly developed yellow color. Each sample was analyzed once per plate, but all plates were run as technical replicates with n=4. Absorption was measured at λ=450 nm and the amount of MPO in samples was calculated from standard curve in SoftmaxPro 6.4 after subtracting the blank. The mean result of this determination was used for subsequent analysis.

Results:

TABLE 5
Plasma concentration, skin concentration, NE activity, wound sizes:

| | Total | Total | NE activity in | |
|---|---|---|---|---|
| | concentration in | concentration | wound tissue | Visual wound |
| | plasma, geo | in skin | [ng/mL | size [% initial |
| | mean [μM]* | [μM] | equivalent] | area at day 8] |
| Vehicle | — | — | 6923 (n = 9) | 94.37 (n = 9) |
| Example 5; 0.03 | 0.0029 (n = 4) | 0.01 (n = 5) | 7850 (n = 4) | 98.5 (n = 4) |
| mg/kg BW, o.d. | | | | |
| Example 5; 0.1 | 0.011 (n = 6) | 0.03 (n = 6) | 8237 (n = 6) | 86.07 (n = 6) |
| mg/kg BW, o.d. | | | | |
| Example 5; 0.3 | 0.027 (n = 8) | 0.06 (n = 8) | 6677 (n = 8) | 72 ± (n = 8) |
| mg/kg BW, o.d. | | | | |
| Example 5; 1.0 | 0.067 (n = 8) | 0.16 (n = 8) | 5018 (n = 8) | 71.16 (n = 8) |
| mg/kg BW, o.d. | | | | |
| Example 5; 3.0 | 0.13 (n = 9) | 0.30 ± 0.06 (n = 9) | 4159 (n = 9) | 68.1 (n = 9) |
| mg/kg BW, o.d. | | | | |
| Example 5; 10.0 | 0.17 (n = 10) | 0.54 (n = 10) | 2177 (n = 10) | 54.85 (n = 10) |
| mg/kg BW, o.d. | | | | |

*unbound conc.: [total]*0.31

A linear increase of exposure was found in blood plasma as well as in skin (both in native as well as wounded skin). This correlated well with an inhition of NE activity in wound tissue which in turn correlated with an improved visual wound size reduction.

TABLE 6
Potencies (EC₅₀) of Example 5 to inhibit NE
activity and improve wound size reduction:

| | EC₅₀ [nM] |
|---|---|
| NE activity | 8.8 |
| Wound size | 4.5 |

The potencies of Example 5 with regard to both NE inhibition and visual wound size reduction were virtually identical.

B-2c) Reduced Neutrophil-Dependent Inflammatory Activity in Wounds of Diabetic Mice (Db/Db) Delayed Healing

To assess neutrophil activity in wound tissue samples, myeloperoxidase activity was measured.

TABLE 7
Neutrophil dependent inflammatory activity
in wound tissue (MPO activity):

| | Peroxidase Activity/μg Protein |
|---|---|
| Vehicle | 0.00274 (n = 10) |
| Example 5; 0.03 mg/kg BW, o.d. | 0.00361 (n = 10) |
| Example 5; 0.1 mg/kg BW, o.d. | 0.00029 (n = 8) |
| Example 5; 0.3 mg/kg BW, o.d. | 0.00043 (n = 10) |
| Example 5; 1.0 mg/kg BW, o.d. | 0.00043 (n = 10) |
| Example 5; 3.0 mg/kg BW, o.d. | 0.00027 (n = 9) |
| Example 5; 10.0 mg/kg BW, o.d. | 0.00016 (n = 10) |

Example 5 potently and effectively inhibited MPO activity in wound tissue already at a low dose of 0.1 mg/kg.

B-2d) Improved Collagen Deposition in Chronic Skin Lesions of Adriamycin-Treated Rats

One of the local complications of some cytotoxic agents is local tissue necrosis due to extravasation. Extravasation of adriamyin causes severe and progressive tissue necrosis and ulceration. These ulcers form slowly and heal with great difficulty. The mechanism of this tissue damage is not entirely clear, but beyond antiproliferative effects, neutrophilic infiltrations may play a prominent role. NE may thus well contribute to healing delay by continued disruption of newly formed ECM molecules.

Materials and Method:

Animals: rats, male. Fischer 344

Weight: 176-200 g on delivery

Breeder: Ch.River Sulzfeld

food: ssniff R/M-H, 10 mm and water ad libitum

TABLE 8

| | Rats/group | Wound | | | |
|---|---|---|---|---|---|
| Group | (n) | stimulator | Example | Dose | Vehicle |
| 1 | 10 | PBS | — | — | DEPO |
| 2 | 10 | Adriamycin | — | — | DEPO |
| 3 | 10 | Adriamycin | Example 5 | 10 mg/kg BW | DEPO |

Compounds:
Adriamycin—i.d. 0.15 mL (2 mg/mL NaCl)
PBS—i.d. 0.15 mL
Example 5

Vehicle:
DEPO—DMSO, Ethanol, Peanut Oil 5/3/92 (V/V/V) o.d., p.o., 5 ml/kg BW

Experimental Procedure:

Day 0:
Animal randomization, marking, inhalation anesthesia: isoflurane, O₂, N₂O mixture, shaving of lower back, the animals remain on warming pads in order to avoid cooling down during the adriamycin injection, pain prevention with Temgesic s.c.

Depot set: 3 intradermal injections of 0.15 ml adriamycin (2 mg/ml) per animal in front and rear back, size measurement of the depot via caliper.

On day 2, 4, 7, 9, 11, 14, 16, 18, 21, 23, 25 and 28
inhalation anesthesia: isoflurane, O₂, N₂O mixture
size measurement of the depot and photos

Day 28 section
inhalation anesthesia: isoflurane, O₂, N₂O mixture under anesthesia and then dislocation of the cervical spine
size measurement of the depot and the incision wound and photos
Former depots were harvested by punch biopsies
Wound 1/custodian of any animal->in liquid N₂ for peroxidase and elastase determination
Wound 2/custodian of any animal->in liquid N₂ for Hydroxyproline determination
Wound 3/custodian of any animal->in formalin fixed for histological studies
Samples were stored for further procedures at −80° C.

Hydroxyproline (HYP) Assay

The insoluble fraction after tissue lysis also contains collagen which can be quantified by measuring the amount of HYP upon hydrolysis with acid or base. The assay is based on the one described by Edwards and O'Brien (Clinica Chimica Acta, 104 (1980) 161-167) with a few modifications. Wound homogenate pellets were placed in 50 mL DigiPrep tubes with 6 mL 6M hydrochloric acid and lids closed tightly. Hydrolysis was done for 16 h at 115° C. (temperature sensor on inner wall) in a DigiPrep heat block. After a brief cool down phase lids were removed and acid evaporated until dryness. Next 2.5 mL of bidest. H₂O was added and samples were allowed to dissolve again for 30 min at RT on a roll mixer. The supernatant was placed in a 96 deep-well MWP and centrifuged at 1000 g for 10 min to remove large debris. The standard curve was prepared with hydroxyproline in bidest. water, which also served as a blank. For the assay 10 μL of each sample, standard and blank were dispensed in a clear flat bottom MWP. Then 90 μL of solution A was added, plates were covered with an adhesive foil and incubated for 25 min at RT. After adding 100 μL of solution B plates were covered with adhesive aluminium foil and incubated for 30 min at 65° C. in an oven. After allowing them to cool down to RT, absorbance was measured at 560 nm in a plate reader. Each sample was analyzed once per plate, but all plates were run as technical replicates with n=4. HYP was quantified against the standard curve after subtracting the blank in SoftmaxPro 6.4. The mean result of this determination was used for subsequent analysis.

For NE and MPO Activity Assay Description See Above.

Paraffin-Embedded Skin Samples:

the skin piece is placed with the downside on a piece of cork and 4 with needles and transferred into formalin (3.7% in PBS). After sufficient time in formalin (24-72 h), the samples were trimmed to an appropriate size and transferred to histology-cassettes. The histo-cassettes are watered 2 h under running tap water at room temperature. The drainage and waxing was done using a Leica ASP200, the samples were embedded in paraffin, sectioned by microtome and mounted on slides. The paraffin sections were stained depending on the question (Ladewig or Sirius Red)

B-2e) Improved Collagen Depostion in Chronic Skin Lesions of Adriamycin-Treated Rats Shown by Ladewig Staining

To assess the content of collagen in ulcer tissue, Ladewig staining was performed according to standard protocols. A scoring system was applied where blueish staining of normal skin tissue was defined “0”. Absence of any blue staining was defined “−10”.

Results

TABLE 9

| | Ladewig | Hydroxy- |
|---|---|---|
| | score | proline |
| Vehicle (PBS) | −2.89 (n = 9) | 5230 (n = 9) |
| Vehicle (PBS) + Adriamycin | −4.44 (n = 9) | 4662 (n = 10) |
| Example 5; 10 mg/kg BW, o.d. + | −1.38 (n = 8) | 5169 (n = 10) |
| Adriamycin | | |

B-2f) Improved Collagen Type III Deposition in Chronic Skin Lesions of Adriamycin-Treated Rats Shown by Sirius Red Staining

To assess the content of collagen type I (mature form) and collagen type III (newly formed) in ulcer tissue, Sirius red staining was performed according to standard protocols.

TABLE 10

| | Collagen | Collagen |
|---|---|---|
| | type I | type III |
| Vehicle (PBS) | 19.15 (n = 8) | 21.16 (n = 8) |
| Vehicle (PBS) + Adriamycin | 7.63 (n = 10) | 17.82 (n = 10) |
| Example 5; 10 mg/kg BW, o.d. + | 9.7 (n = 7) | 46.69 (n = 7) |
| Adriamycin (d 14-28) | | |
| Example 5; 10 mg/kg BW, o.d. + | 8.8 (n = 8) | 72.23 (n = 8) |
| Adriamycin (d 21-37) | | |

While collagen type I was reduced in Adriamycin-induced lesions and not reestablished after 28 or 37 days post wounding, there was a strong increase of collagen type III in Adriamycin-induced lesions after two weeks of treatment with Example 5.

B-22) Improved Collagen Type III Deposition in Lesions of Pressure-Applied Mouse Skin Shown by Sirius Red Staining

OBJECTIVE: Pressure ulcers (decubitus ulcers) represents a large subgroup of chronic wounds. They mainly affect aged or neurologically immobilized patients. Continuous pressure at discrete skin areas leads to long-lasting hard-to-heal ulcer wounds that are also characterized by neutrophil infiltration. The presented experiment was set up to mimick this condition by applying magnet-induced pressure onto mouse skin folds. Since the area of emerging wounds is variable in form and size, its usability as a readout parameter is technically limited. This study thus focused on Collagen neogenesis after pressure ulcer induction with or without treatment with the compound of Example 5.

METHODS: Magnet placement leads to wounds with dermal neutrophil infiltration and delayed wound healing. Three ischemia-reperfusion (IR) cycles (12 hours on, 12 hours off) were applied in each animal to initiate decubitus ulcer formation.

Materials and Method:

Animals: mice, male. Balb/c

Age: 8 weeks on delivery

Breeder: Ch.River Sulzfeld

food: ssniff R/M-H, 10 mm and water ad libitum

Magnets: 5*12 mm disc magnets (Supermagnet Inc.)

On day 0 (start of experiment), the mice were weighed and put in anesthesia using an isoflurane/O₂/N₂O mixture. Back and flank skin was shaven. Skin was moistened with tap water and depilated using Pilca cream. Skin was lifted at the back midline and one magnet was placed on each side of the skin fold so that they were exactly opposite to each other. The magnets were left in place for 12 hours, then taken off for 12 hours. This cycle was repeated three times in total. From day 3 to day 15, mice were treated with the compound of Example 5 once daily intragastrally.

After sacrificing (day 15) wounds were excised using a 10 mm punch scalpel.

To assess the content of collagen type I (mature form) and collagen type III (newly formed) in ulcer tissue, Sirius red staining was performed according to standard protocols.

TABLE 11

| | Mice/group | Wound | | | |
|---|---|---|---|---|---|
| Group | (n) | stimulator | Example | Dose | Vehicle |
| 1 | 10 | Pressure | — | — | DEPO |
| 2 | 10 | Pressure | Example 5 | 1 mg/kg BW | DEPO |
| 3 | 10 | Pressure | Example 5 | 10 mg/kg BW | DEPO |

Compounds: Example 5

Vehicle:

DEPO—DMSO, Ethanol, Peanut Oil 5/3/92 (VNN) o.d., p.o., 5 ml/kg BW

Results

TABLE 12

| | Collagen | Collagen |
|---|---|---|
| | type I | type III |
| Vehicle (PBS) + pressure | 0.6 (n = 8) | 23.2 (n = 8) |
| Example 5; 1 mg/kg BW, o.d. + pressure | 1.1 (n = 9) | 30.2 (n = 9) |
| Example 5; 10 mg/kg BW, o.d. + pressure | 0.7 (n = 7) | 49.8 (n = 7) |

While collagen type I was strongly reduced in pressure-induced lesions and was not reestablished after 12 days post pressure, there was a significant increase of collagen type III in pressure-induced lesions after 12 days of treatment with Example 5.

### EXPLANATION OF THE FIGURES

FIG. 1: B-2a) Visual healing (planometric assessment) of full thickness skin excision wounds in Tsk mice (wounds with solid eschar were excluded).

FIG. 2: B-2g) Improved collagen type III deposition in lesions of pressure-applied mouse skin shown by Sirius Red staining

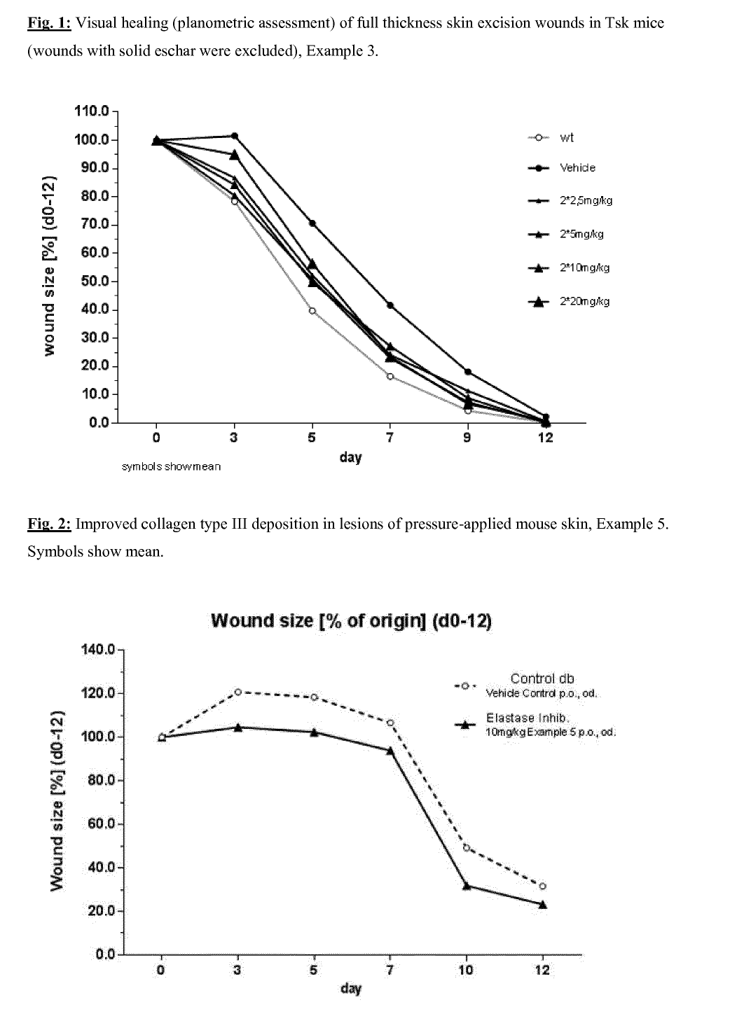

### B) WORKING EXAMPLES OF PHARMACEUTICAL COMPOSITIONS

The substances according to the invention can be converted to pharmaceutical preparations as follows:

Tablet:

Composition:

100 mg of the compound of Example 1, 50 mg of lactose (monohydrate), 50 mg of maize starch, 10 mg of polyvinylpyrrolidone (PVP 25) (from BASF, Germany) and 2 mg of magnesium stearate.

Tablet weight 212 mg. Diameter 8 mm, radius of curvature 12 mm.

Production:

The mixture of the compound of Example 1, lactose and starch is granulated with a 5% strength solution (m/m) of the PVP in water. After drying, the granules are mixed with the magnesium stearate for 5 min. This mixture is compressed in a conventional tablet press (see above for format of the tablet).

Oral Suspension:

Composition:

1000 mg of the compound of Example 1, 1000 mg of ethanol (96%), 400 mg of Rhodigel (xanthan gum) (from FMC, USA) and 99 g of water.

10 ml of oral suspension correspond to a single dose of 100 mg of the compound according to the invention.

Production:

The Rhodigel is suspended in ethanol, and the compound of Example 1 is added to the suspension. The water is added while stirring. The mixture is stirred for approx. 6 h until the Rhodigel has finished swelling.

Intravenously Administrable Solution:

Composition:

1 mg of the compound of Example 1, 15 g of polyethylene glycol 400 and 250 g of water for injection purposes.

Production:

The compound of Example 1 is dissolved together with polyethylene glycol 400 by stirring in the water. The solution is sterilized by filtration (pore diameter 0.22 μm) and dispensed under aseptic conditions into heat-sterilized infusion bottles. The latter are closed with infusion stoppers and crimped caps.

Topically Administrable Form

Wound Dressings Such as Gels, Foams, Creams, Ointments

1. Compound of the formula (I) 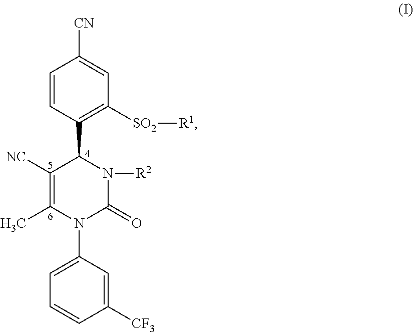
in which
R¹ represents (C₁-C₄)-alkyl, and
R² represents hydrogen, (C₁-C₂)-alkyl or a group of the formula —CH₂—C(═O)—NH—R³ or —SO₂—R⁴, in which
R³ represents hydrogen or (C₁-C₂)-alkyl and
R⁴ represents (C₁-C₂)-alkyl or (C₃-C₄)-cycloalkyl,
or a salt, a solvate or a solvate of a salt thereof,
for use in a method for the treatment and/or recurrence rate reduction of a chronic wound selected from the group consisting of a pressure ulcer, a diabetic ulcer on the extremities, a venous leg ulcer, an arterial leg ulcer, a mixed leg ulcer, and a chronic wound associated with Behçet's disease, wherein the compound of the formula (I) is administered orally and wherein the treatment and/or recurrence rate reduction of the chronic wound causes one or more of the effects selected from an increased wound closure rate, a reduced wound size, a shorter time to wound closure, an increase of the reepithelialisation of the chronic wound, an increase of the deposition of extracellular matrix such as collagen in the chronic wound, and a reduction of pain related to the chronic wound.

2. Compound of the formula (I) as defined in claim 1 or a salt, a solvate or a solvate of a salt thereof, for use in a method for the treatment of neutrophilic dermatoses selected from Behçet's disease, PAPA-syndrome, PASH syndrome, SAPHO syndrome, and subcorneal pustular dermatosis or for use in a method for the treatment of autoimmune blistering dermatoses.

3. Compound of the formula (I) as defined in claim 1 for use in a method as defined in claim 2, wherein the autoimmune blistering dermatoses are selected from the group consisting of pemphigus, bullous pemphigoid, epidermolysis bullosa acquisita, mucous membrane pemphigoid, pemphigoid gestationis, linear IgA dermatosis, and dermatitis herpetiformis.

4. Compound of the formula (I) as defined in claim 1, in which
R¹ represents (C₁-C₂)-alkyl, and
R² represents hydrogen, methyl or a group of the formula —CH₂—C(═O)—NH—R³ or —SO₂—R⁴, in which
R³ represents hydrogen or methyl and
R⁴ represents methyl or cyclopropyl,
or a salt, a solvate or a solvate of a salt thereof, for use in a method according to any of claims 1 to 3.

5. Compound of the formula (I) as defined in claim 1 or 4, wherein the compound is selected from the group consisting of
(4S)-4-[4-Cyano-2-(methylsulfonyl)phenyl]-6-methyl-2-oxo-1-[3-(trifluoromethyl)phenyl]-1,2,3,4-tetrahydropyrimidine-5-carbonitrile,
2-[(6S)-5-Cyano-6-[4-cyano-2-(methylsulfonyl)phenyl]-4-methyl-2-oxo-3-[3-(trifluoromethyl)-phenyl]-3,6-dihydropyrimidin-1(2H)-yl]acetamide,
(4S)-4-[4-Cyano-2-(methylsulfonyl)phenyl]-6-methyl-3-(methylsulfonyl)-2-oxo-1-[3-(trifluoromethyl)phenyl]-1,2,3,4-tetrahydropyrimidine-5-carbonitrile,
(4S)-4-[4-Cyano-2-(methylsulfonyl)phenyl]-6-methyl-3-(cyclopropylsulfonyl)-2-oxo-1-[3-(tri-fluoromethyl)phenyl]-1,2,3,4-tetrahydropyrimidine-5-carbonitrile,
(4S)-4-[4-Cyano-2-(methylsulfonyl)phenyl]-3,6-dimethyl-2-oxo-1-[3-(trifluoromethyl)phenyl]-1,2,3,4-tetrahydropyrimidine-5-carbonitrile,
(4S)-4-[4-Cyano-2-(ethylsulfonyl)phenyl]-3,6-dimethyl-2-oxo-1-[3-(trifluoromethyl)phenyl]-1,2,3,4-tetrahydropyrimidine-5-carbonitrile, and
or a salt, a solvate or a solvate of a salt thereof, for use in the method according to any of claims 1 to 3.

6. Compound of the formula (I) as defined in claim 1, 4, or 5, wherein the compound is selected from the group consisting of
(4S)-4-[4-Cyano-2-(methylsulfonyl)phenyl]-6-methyl-3-(methylsulfonyl)-2-oxo-1-[3-(trifluoromethyl)phenyl]-1,2,3,4-tetrahydropyrimidine-5-carbonitrile and
(4S)-4-[4-Cyano-2-(methylsulfonyl)phenyl]-3,6-dimethyl-2-oxo-1-[3-(trifluoromethyl)phenyl]-1,2,3,4-tetrahydropyrimidine-5-carbonitrile,
or a salt, a solvate or a solvate of a salt thereof, for use in the method according to any of claims 1 to 3.

7. (4S)-4-[4-Cyano-2-(methylsulfonyl)phenyl]-3,6-dimethyl-2-oxo-1-[3-(trifluoromethyl)phenyl]-1,2,3,4-tetrahydropyrimidine-5-carbonitrile or a salt, a solvate or a solvate of a salt thereof, for use in the method according to any of claims 1 to 3.

8. Compound of the formula (I) as defined in any of claims 1 and 4 to 7 for use in the method according to any of claims 1 to 3, wherein the compound of formula (I) is administered either alone or in addition to one or more of the physical, biological, topical and/or systemic wound management therapies selected from the group consisting of topical wound dressings, topical antiseptics, wound excision or debridement, weight reduction, appropriate footwear for an offloading effect, PDGF (Regranex), hyperbaric oxygen therapy, compression therapy wound therapy with negative pressure, maggot debridement therapy, and therapy with systemic antibiotics, wherein the physical and/or topical wound management therapies are employed simultaneously, sequentially or separately to administering of the compound of formula (I).

9. Medicament, comprising a compound of the formula (I) as defined in any of claims 1 and 4 to 7 in combination with an inert, non-toxic, pharmaceutically suitable auxiliary for the treatment and/or recurrence rate reduction of a chronic wound selected from the group consisting of a pressure ulcer, a diabetic ulcer on the extremities, a venous leg ulcer, an arterial leg ulcer, a mixed leg ulcer, and a chronic wound associated with Behçet's disease, wherein the compound of the formula (I) is administered orally and wherein the treatment and/or recurrence rate reduction of the chronic wound causes one or more of the effects selected from an increased wound closure rate, a reduced wound size, a shorter time to wound closure, an increase of the reepithelialisation of the chronic wound, an increase of the deposition of extracellular matrix such as collagen in the chronic wound, and a reduction of pain related to the chronic wound.

10. Medicament, comprising a compound of the formula (I) as defined in any of claims 1 and 4 to 7 in combination with an inert, non-toxic, pharmaceutically suitable auxiliary for use in a method for the treatment of neutrophilic dermatoses selected from Behçet's disease, PAPA-syndrome, PASH syndrome, SAPHO syndrome, and subcorneal pustular dermatosis or for use in a method for the treatment of autoimmune blistering dermatoses selected from the group consisting of pemphigus, bullous pemphigoid, epidermolysis bullosa acquisita, mucous membrane pemphigoid, pemphigoid gestationis, linear IgA dermatosis, and dermatitis herpetiformis.

11. Medicament, comprising a compound of the formula (I) as defined in any of claims 1 and 4 to 7 in combination with a further active compound selected from the group consisting of lipid metabolism-modulating active compounds, antidiabetics, perfusion-enhancing and/or antithrombotic agents and also antioxidants, aldosterone and mineralocorticoide receptor antagonists, vasopressin receptor antagonists, organic nitrates and NO donors, IP receptor agonists, EP receptor agonists and antagonists, positive inotropic compounds, ACE inhibitors, cGMP- and cAMP-modulating compounds, natriuretic peptides, NO-independent stimulators of guanylate cyclase, NO-independent activators of guanylate cyclase, compounds which inhibit proinflammatory signal transduction cascades, soluble guanylate cyclase (sGC) stimulators or inhibitors, chemokine receptor antagonists, p38 kinase inhibitors, NPY agonists, orexin agonists, anorectics, PAF-AH inhibitors, antiphlogistics, analgesics, AR alpha 2c antagonists, MMP inhibitors, glucocorticoid receptor agonists HIF PH inhibitors, oxidative stress modulators, and pH modulators, systemic or intra/perilesional applied growth factors or systems consisting of living keratinocytes and/or growth factors, topically applied, e.g. as foam or spray, for the treatment and/or recurrence rate reduction of a chronic wound selected from the group consisting of a pressure ulcer, a diabetic ulcer on the extremities, a venous leg ulcer, an arterial leg ulcer, a mixed leg ulcer, and a chronic wound associated with Behçet's disease, wherein the compound of the formula (I) is administered orally and wherein the treatment of the chronic wound causes one or more of the effects selected from increased wound closure rates, shorter time to wound closure, an increase of the reepithelialisation of the chronic wound, an increase of the deposition of extracellular matrix such as collagen in the chronic wound, and a reduction of pain related to the chronic wound.

12. Medicament, comprising a compound of the formula (I) as defined in any of claims 1 and 4 to 7 in combination with a further active compound selected from the group consisting of lipid metabolism-modulating active compounds, antidiabetics, perfusion-enhancing and/or antithrombotic agents and also antioxidants, aldosterone and mineralocorticoide receptor antagonists, vasopressin receptor antagonists, organic nitrates and NO donors, IP receptor agonists, EP receptor agonists and antagonists, positive inotropic compounds, ACE inhibitors, cGMP- and cAMP-modulating compounds, natriuretic peptides, NO-independent stimulators of guanylate cyclase, NO-independent activators of guanylate cyclase, compounds which inhibit proinflammatory signal transduction cascades, soluble guanylate cyclase (sGC) stimulators or inhibitors, chemokine receptor antagonists, p38 kinase inhibitors, NPY agonists, orexin agonists, anorectics, PAF-AH inhibitors, antiphlogistics, analgesics, AR alpha 2c antagonists, MMP inhibitors, glucocorticoid receptor agonists HIF PH inhibitors, oxidative stress modulators, and pH modulators, systemic or intra/perilesional applied growth factors or systems consisting of living keratinocytes and/or growth factors, topically applied, e.g. as foam or spray, for the treatment of neutrophilic dermatoses selected from Behçet's disease, PAPA-syndrome, PASH syndrome, SAPHO syndrome, and subcorneal pustular dermatosis or for use in a method for the treatment of autoimmune blistering dermatoses selected from the group consisting of pemphigus, bullous pemphigoid, epidermolysis bullosa acquisita, mucous membrane pemphigoid, pemphigoid gestationis, linear IgA dermatosis, and dermatitis herpetiformis.

13. Method for the treatment and/or recurrence rate reduction of a chronic wound selected from the group consisting of a pressure ulcer, a diabetic ulcer on the extremities, a venous leg ulcer, an arterial leg ulcer, a mixed leg ulcer, and a chronic wound associated with Behçet's disease, wherein the compound of the formula (I) is administered orally and wherein an effective amount of at least one compound of the formula (I) as defined in any of claims 1 and 4 to 7 or a medicament as defined in claim 9 or 11 is administered orally to a patient in need thereof and wherein the treatment of the chronic wound causes one or more of the effects selected from an increased wound closure rate, a reduced wound size, a shorter time to wound closure, an increase of the reepithelialisation of the chronic wound, an increase of the deposition of extracellular matrix such as collagen in the chronic wound, and a reduction of pain related to the chronic wound.

14. Method for the treatment of neutrophilic dermatoses selected from Behçet's disease, PAPA-syndrome, PASH syndrome, SAPHO syndrome, and subcorneal pustular dermatosis or for use in a method for the treatment of autoimmune blistering dermatoses selected from pemphigus, bullous pemphigoid, epidermolysis bullosa acquisita, mucous membrane pemphigoid, pemphigoid gestationis, linear IgA dermatosis, and dermatitis herpetiformis, wherein an effective amount of at least one compound of the formula (I) as defined in any of claims 1 and 4 to 7 or a medicament as defined in claim 10 or 12 is administered to a patient in need thereof.
